SEQUENCE LISTING (24 sequences)

SEQ ID NO: 1 - DNA, 2423 bp, Homo Sapiens
acccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgtctgagtctgcagtcccgagatcccagccatcatgtccatagagaagatctgggcccgggagatcctggactcccgcgggaaccccacagtggaggtggatctctatactgccaaaggtcttttccgggctgcagtgcccagtggagcctctacgggcatctatgaggccctggagctgagggatggagacaaacagcgttacttaggcaaaggtgtcctgaaggcagtggaccacatcaactccaccatcgcgccagccctcatcagctcaggtctctctgtggtggagcaagagaaactggacaacctgatgctggagttggatgggactgagaacaaatccaagtttggggccaatgccatcctgggtgtgtctctggccgtgtgtaaggcaggggcagctgagcgggaactgcccctgtatcgccacattgctcagctggccgggaactcagacctcatcctgcctgtgccggccttcaacgtgatcaatggtggctctcatgctggcaacaagctggccatgcaggagttcatgatcctcccagtgggagctgagagctttcgggatgccatgcgactaggtgcagaggtctaccatacactcaagggagtcatcaaggacaaatacggcaaggatgccaccaatgtgggggatgaaggtggctttgcccccaatatcctggagaacagtgaagccttggagctggtgaaggaagccatcgacaaggctggctacacggaaaagatcgttattggcatggatgttgctgcctcagagttttatcgtgatggcaaatatgacttggacttcaagtctcccactgatccttcccgatacatcactggggaccagctgggggcactctaccaggactttgtcagggactatcctgtggtctccattgaggacccatttgaccaggatgattgggctgcctggtccaagttcacagccaatgtagggatccagattgtgggtgatgacctgacagtgaccaacccaaaacgtattgagcgggcagtggaagaaaaggcctgcaactgtctgctgctcaaggtcaaccagatcggctcggtcactgaagccatccaagcgtgcaagctggcccaggagaatggctggggggtcatggtgagtcatcgctcaggagagactgaggacacattcattgctgacctggtggtggggctgtgcacaggccagatcaagactggtgccccgtgccgttctgaacgtctggctaaatacaaccagctcatgagaattgaggaagagctgggggatgaagctcgctttgccggacataacttccgtaatcccagtgtgctgtgattcctctgcttgcctggagacgtggaacctctgtctcatcctcctggaaccttgctgtcctgatctgtgatagttcaccccctgagatcccctgagccccagggtgcccagaacttccctgattgacctgctccgctgctccttggcttacctgacctcttgctgtctctgctcgccctcctttctgtgccctactcattggggttccgcactttccacttcttcctttctctttctctcttccctcagaaactagaaatgtgaatgaggattattataaaagggggtccgtggaagaatgatcagcatctgtgatgggagcgtcagggttggtgtgctgaggtgttagagagggaccatgtgtcacttgtgctttgctcttgtcccacgtgtcttccactttgcatatgagccgtgaactgtgcatagtgctgggatggaggggagtgttgggcatgtgatcacgcctggctaataaggctttagtgtatttatttatttatttattttatttgtttttcattcatcccattaatcatttccccataactcaatggcctaaaactggcctgacttgggggaacgatgtgtctgtatttcatgtggctgtagatcccaagatgactggggtgggaggtcttgctagaatgggaagggtcatagaaagggccttgacatcagttcctttgtgtgtactcactgaagcctgcgttggtccagagcggaggctgtgtgcctgggggagttttcctctatacatctctccccaaccctaggttccctgttcttcctccagctgcaccagagcaacctctcactccccatgccacgttccacagttgccaccacctctgtggcattgaaatgagcacctccattaaagtctgaatcagtgc

SEQ ID NO: 2 - DNA, 10905 bp, homo sapiens
ctgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgtctgagtctgcagtcccgaggtgaagcccccgccaggcccagagcccctgtggccccctcccttcgccgcggcgcccctgcctcctttacccggtgccgctgcgcacctctccgcatctctggcccggtgcagctgcgcacctgctccgccgcccgcgcccagggcgccttccctccctggccttccccgcccgctgccttcgctttctggccctctgcgcgcctatctctaactgcgcctctccacccttgcctgcctctctcccggtgctcgccctcatctacgtctattgttctaagttggacgtctccggcgtgcattttatcctagagcgtcccttttggtttgcatttgggaaatgtcttctctcaccgttcctcacctcccccagacttcccttgactcccctctctcctgctcttccccacggcgcccctctccgttcgcgcttcctcccctctgctgcacgggggagagatggaagaagtggggatctgtcgaggcgcagaggggaggacaggcccagctcgcctccactccccaccggctcttatcctcttcacttcccgctgcaacccccagggactgcagggcctttctcaggaccctcttccccgacacctgtattgcatgcgcctttcgcgggaagaggagggatacacgtttgggagagagtgggaccttggggaaggggcagtgttcaggcagctggggtgttaagttggggaggtatgggggctctggaagagaggcccaggcagcccatcctcttcttggctcccaggagaaatgcaagcctggcagccattccgctctgaggagatctgggaacccaccggctggccaagctgcaaagaggcgcgggaacatgtcgtgccctccctcctcactgcagtgttctccatctcatcatttgatcttacccgcccgggactgcagtgacctccccttcctcactgcattgaccttctcctcttccaggggggagggggaatcccttctacagccctagactgcagccagcgcctccctacccaccccccacccactgcagtaacctctttcccatccctcccagcctcccgccccgcccattgatctcaggctccacccctctaagcctcttatctttctccttccttcccttccacccgaggagatcccagccatcatgtccatagagaagatctgggcccgggagatcctggactcccgcgggaaccccacagtggaggtggatctctatactgccaaaggtaatgggtgtggcatgggccttcctacagccctagcttttccacggccaggctgggttcggccaggggttcggaggccttttttgatacccaggggtatggggtgctgggccaggctcacaagcctgggttgtggcggttggatcctccttgtccgggagccagggtaggtgggtctgtgttcgagtttagcgtgtgagcatgtcctgcctccgtgtgtctgcctgtgcacttgcatgtgtgcagacgtgtgctgcaagcaattttctttctgcgggctcccacttgtgcatgtggggcctcagatgggtggatgaggaggccacttcttgtgcatctgcctcagtgtgtgtgtggggtgggggtggggggatgttagggagcagtgtggggagagagctagatgtggtaggcaggcagtttagagccagaaggctgaaggactccctggcccctgtgtcctcttcactccctctcattccatgttcctcctttaggtcttttccgggctgcagtgcccagtggagcctctacgggcatctatgaggccctggagctgagggatggagacaaacagcgttacttaggcaaaggtgaggtcccttctcttttccagactctcccccacctcagccttatgcccctacctcacaccagtccccagtcctcctctagcatggcttcccctcctcccattgatcccttccgcccctcctggcccgacccagtccagcctcttcctttccccaggtgtcctgaaggcagtggaccacatcaactccaccatcgcgccagccctcatcagctcagtgaggcctgctctttgctggggatagcagggccagagttctggaaggaatcccggagcagggcaggaggaagggaagaaagaaggcccactcttaggaatcatggttacaagggggaagggtggggaacagcttccttaatgcaccctgctcccatgggagttcaggtcccctaatccaggtaggcccctgtcacagggacctggttggaccctggccaaatgtgagcttgggtgtgaatgaggggaccctctgccttaggctcagcctccagcctggccctgggtgatggagctctgccctcagggtctctctgtggtggagcaagagaaactggacaacctgatgctggagttggatgggactgagaacaaatgtgagccgggccggagaaagtgggaagcgtcagggtggggaggcgtggagcagatagagagctgaagggccagtgctgtagtggcttcctcaggaatgactgtcaggggcattctcctctcaaagccagagcaaggggagatgagtttagctgcagagggaaggaccgacagtaggcagaaggaagaccttctttgcagcatacagaggagggggatggcctgagagagtctgtggtctcagggatatttagaaagaggtgtggctctctgccgtttccaatctcctcctccccacccattcctctccctgctgttttcaagagagcatggatgaggtgttgctggggcaggggtggggagggaggagggggtctcctttactggctccttttggagactacagatggaggcaggagctagaaaggagaaggggacatttggctcagcaccttcctctataatctcctagccaagtttggggccaatgccatcctgggtgtgtctctggccgtgtgtaaggcaggggcagctgagcgggaactgcccctgtatcgccacattgctcagctggccgggaactcagacctcatcctgcctgtgccggtgagcaataagccacggtgcggctctcccaggggcgggtgggggagggagcatgcaactcatgaggaatgatgggaggaaagtgaattgagggaggtaaagaggaaggatggggacgtgagacttagtccggaaagctgggggaagtttgggatcttgggttaacactcctggggcgggcagggaggggctctttgacccttctgtctttctgtggctccccaggccttcaacgtgatcaatggtggctctcatgctggcaacaagctggccatgcaggagttcatgatcctcccagtgggagctgagagctttcgggatgccatgcgactaggtgcagaggtctaccatacactcaagggagtcatcaaggacaaatacggcaaggatgccaccaatgtgggggatgaaggtggctttgcccccaatatcctggagaacagtgaaggtgaggccaggagccccactcccagcgtgctaagtcttaccctattgtgggacatcagaaagggtgacacagttcaccaagtcctgagtaggcgtggagggtcctaggactctgcaaactccaaaaggtaccagttcttagagtggattgcagagagcctgccaaattcacatgcagacctagggggacagtatttttttttttttttgagacggagtcttgctctgtcacccaggctggagtgcagtggctcgatctcagctcactgcaacgtccgcctcccgggttcacgccattctcctgcctcagcctcccgagtagctgggactacaggcgcccgccaccactcccggctaattttttgtattttttagtagagacagggtttcactgtggtctcgaactcctgaccttgtgatcggcccgcctcggcctcccaaagtgctgggattacaagcgtgagccaccacgcctggccaggggacagtcttttacctgcctagccagatgtgttagcatctgttaagttgccactggaaggccgggcgcggtggctcacacctgtaatcccagcactttgggaggctgaggcgggtggatcacctgaggtcaggagtttgagaccagcctggccaacaaggtgaaaccccgtctctactaaaaatacaaaaattagccgggcatggtggcgtgtgcctgtaatcacagatactagcggggctgaggcaggaggatcgcttgtacccgggaggcggaggttgtggtgagccgagatcatgccactgcactccagcctgggcaacagagcgagctccgtctcaaaaaaaaaaaaaaaaaaaaaagttgtcactggagcccttgggaatactgggagatggtctggatgacctgttggattcatcccatcccattcgaaatctgtcctgtccccgtcccaacctcctaggcctctagaatccctaatttttctgtgccttgggggaaactgtatagggaatggaaagaatataggtagtggttaagagttaaggttctggggccaaataacctggatttatttgaaccttgacttgtggagttactgctggtgaactttcttacttctctctggaaataataacagaatctagctcatggtagtgtgaggcttaaatgaaatatatataaaatgcttagatgacatgataccaatgaaagtatagtaagtattattaagagaattccattcctctgtgttcctagaagatggcctctcctccagacctgggataaccccaacagcatccccgccacacttccctcaggaacagacctccacctctgccctgaatgtcttttctttccctcctcctccttgcccatccctcctgcttgtactataatctcactgtattctgtccccagccttggagctggtgaaggaagccatcgacaaggctggctacacggaaaagatcgttattggcatggatgttgctgcctcagagttttatcgtgatggcaaatatgacttggacttcaagtctcccactgatccttcccgatacatcactggggaccagctgggggcactctaccaggactttgtcagggactatcctggtgagaggaagtggtgtgagggggaggtctgggggcaggcagggacgtgtcccagcaactctggaccttatggggtgctgactcaggcaccaggtgggggtgtcctaagaagaacctgagaaccagggagagggtgcaggagccacctgcaaagactgggctttgtatgtagtgtaaaaaatgcaggtacccgtgaccaatctgttctgtctcagatcctgattaaagtcatgggttctgaaaactactggggtcatggggaaggctctggaaggaaccagggatgatatgagtgtgactggatctagcagagaactagaacatttcagtatctttgattgatgaaattgtggatgctgaatggagctgggactgatgtgtgagtagaaagaaggctgagggggactgaattagcttctgcaagtctgcccagggcctttatctcaggatggaggcagttggtcgccatcttcctgacacagagcgaaagaaaatcaacataattgcagaattaaggatttggattagcaatgaggaagggcttgctggcagtgagaacaggaaaatgcaaagctaggttgtgaacccttgttactggaagattttttttggggggggggtggggttgttttttttgttttgttttttttttttgagacagagtttgactcttcttgcccaggctggagtgcaatggcgccatctcggctcaccacaacctccactcccggattcaagcgattcttctgcctcagcctcccgaatagctgggattacagcatgtgccaccatgcctggctaattttgtatttttagtagagacaggggtttctccatgttggtcaggctggtctccaactcctgacctcaggtaatctatccaccttggtctcccaaagtgctgggattacagcgtgagccaccacgcctggctggtttgcttttaattaactttttttttttttttttttttttgtagagacagggtttaccatgttgctcatggctggtctcaaactactgggctcaagtgatctgcatgcctcggcctcccaaagtgctgggattacaggcatgagccactgtgcccagcctatggaagtggcgtgagatctctgctcactgcacgattctcttagcctcccaagtagctgggattacaggcgtgcaccaccatgcctgctaatttttgtatttttagtagagatggggttttactatgttagccagggaactcctatcctcaagtgatccgttcacctcagtatcccaaagtgctgggattacaggcatgagccactgtgcctggcctctccatgtaaggttttatgaaataagaatcaggagccaggcgtggtgctcatgcctgtaatcccattactttgggaagccgaggcaggaggactgcttgaatccaggagttcgagactggcctggcaatacagtgagacctcatctctacaaaaattttaaaaattagctgagtgtggtgccacacacctaaagtccctgctactcaggaggctgaggtggcaggatcacttgattggggaggtggaggttgcagtgagctgagattgtgccactgcactccagcctggatgacagaatgaggctctgtcaaaaaaaaaaaaaaagttaagaatcagttagggcaggcttacactggggggatttgtcttagcaaggatgagcaggtgtagttaaccaagggcctgtccatttcagggaataaaggggcatgttcctgcctgatgtagagacccagggaagatgaacacctcccccctccccaccatgctctctctgcagtggtctccattgaggacccatttgaccaggatgattgggctgcctggtccaagttcacagccaatgtagggatccagattgtgggtgatgacctgacagtgaccaacccaaaacgtattgagcgggcagtggaagaaaaggcctgcaactgtctgctgctcaaggtcaaccagatcggctcggtcactgaagccatccaagcgtgagtgacttctggccctctcctgtgtggtcctcgtttctataagactccttttgcaagtgctccagcctaattctacccaggggtgccaaagagagcggggaacctggaatcatcctcacagttctctcacctctgcccctccacccctgattctctgctcccctcccagatagctttcccctagatgtttcctgacatagaccaaggttggggctgggaagagagtgcccagtgtgagagctggagaatcagtgctgtgtgtggatacaggtgcaagctggcccaggagaatggctggggggtcatggtgagtcatcgctcaggagagactgaggacacattcattgctgacctggtggtggggctgtgcacaggccaggtgagtgaggcagcctggtgagtgaagagaactctctgtgggattggtatttctagctcacccacctggtctctccttccaggtgtttgagggtgtcaggggagtttcaggagagcagaagtttcctttcaggggtgagagggcagtcactgagctgcaaatcctttgaaatgtttcagatcaagactggtgccccgtgccgttctgaacgtctggctaaatacaaccagctcatgaggtgagggtccctggggtgggagcccctggcccagatggctaaaggccccatttgcctgccagaccatctgtagcaccaagggcctggataacagtccatttcctggataacagtccaacagataatattggtttttgcttcctgggtttattgatggcctgattgacaaatcccagagatcacatgggaaagccagggaatgctaagccttggggcaggacacaaaagcaggtggtgtgggggtggttggagtctgggggacccctagagagagaagcaggatcctcctgcatccctgaccacttcctttgtggttcatctctctcagaattgaggaagagctgggggatgaagctcgctttgccggacataacttccgtaatcccagtgtgctgtgattcctctgcttgcctggagacgtggaacctctgtctcatcctcctggaaccttgctgtcctgatctgtgatagttcaccccctgagatcccctgagccccagggtgcccagaacttccctgattgacctgctccgctgctccttggcttacctgacctcttgctgtctctgctcgccctcctttctgtgccctactcattggggttccgcactttccacttcttcctttctctttctctcttccctcagaaactagaaatgtgaatgaggattattataaaagggggtccgtggaagaatgatcagcatctgtgatgggagcgtcagggttggtgtgctgaggtgttagagagggaccatgtgtcacttgtgctttgctcttgtcccacgtgtcttccactttgcatatgagccgtgaactgtgcatagtgctgggatggaggggagtgttgggcatgtgatcacgcctggctaataaggctttagtgtatttatttatttatttattttatttgtttttcattcatcccattaatcatttccccataactcaatggcctaaaactggcctgacttgggggaacgatgtgtctgtatttcatgtggctgtagatcccaagatgactggggtgggaggtcttgctagaatgggaagggtcatagaaagggccttgacatcagttcctttgtgtgtactcactgaagcctgcgttggtccagagcggaggctgtgtgcctgggggagttttcctctatacatctctccccaaccctaggttccctgttcttcctccagctgcaccagagcaacctctcactccccatgccacgttccacagttgccaccacctctgtggcattgaaatgagcacctccattaaagtctgaatcagtgcactgttgtgtctaaggagtcttactctagtccctatgaggggagagaagatggagcacctggaagctggtgaaactggatagcagagctgggggggcacaaaaagaggaagacaaactgaacaaatatggccgagatgatggcactgcctaccccattctggctaggtggggtgcatgtggcccctgctttcttagcagaaggcttggctcccagacgcaggtgaattaaggggttcaagagcccctaaaagcataaaatattttgtgtgtgtgtgtgtgtgcacgcgcattttgggggaaagggggtctaaggtgttttcatatccaaagggcttgtggactggagcagctcctgtactgggcctctgccaacaaaaccctggctggttctcgaatggaacaggacttcatggccatcacccactgcaagatggggaaatgggaaggaagaatggttccgggggtagtatacggaaggacctaaggaaacagagtcctcaataaactgaagattcaggaacaaaagtgcttaacagaaccctggctgggtcagactaacagtaggtttccaatatgtggctagagacgtac

SEQ ID NO: 3 - DNA, 2218 bp, Rattus Sp.
cgccgccgccgtcaccaccgccaccgccaccggctgagtctgcagtcctcgaggagatcccagccatcatgtctatacagaagatctgggcccgagagatcttggactcccgtgggaatcccaccgtggaggtggatctccatactgccaaaggtcttttccgggctgcagtccccagtggggcctccactggcatctatgaggccctggagctaagggatggggacaaacagcgttacttaggcaaaggtgtcctgaaggctgtggaccacatcaacagcaccatcgcaccggccctcatcagctcaggcctctctgtggtggagcaggagaagctggacaacctgatgctggagttggatgggactgagaacaaatccaagtttggggccaatgccatcctgggtgtgtccctggccgtgtgcaaggctggggcagccgagaaggacttgcccctctatcgccacattgctcaactggctgggaactccgacctcatcctgcccgtgccggcctttaatgtgatcaacggtggctctcatgctgggaacaagttggccatgcaggagttcatgatcctcccagtgggtgctgagagctttcgggatgccatgcgacttggggccgaggtgtaccacacactcaagggggtcatcaaggacaagtacggcaaggatgccactaatgtgggggatgaaggcggctttgcccccaatatcctggagaacagcgaagctttggagctggtgaaggaagccattgacaaggctggctacacggaaaagatggtgattggtatggatgtggctgcctctgagttttaccgcgatggcaaatacgacttggatttcaagtctcctgctgacccttcccgatgcatcactggggaccagcttggggcactctaccaggactttgtccggaactatcctgtggtctccattgaagacccattcgaccaggatgactgggcagcttggtccaagttcacagccaatgtcggcatccagatagtgggtgatgacctgacggtgaccaaccccaagcgcatcgagcgggcagtggaggagaaggcctgcaactgtttgctgctcaaggtcaaccagatcggctcagtcacagaagccatccaagcgtgcaagctggcccaggagaacggctggggggttatggtgagtcatcgctctggagaaaccgaggacacgttcattgcagacctcgtagtgggactgtgtacaggtcagatcaagactggtgccccatgcagatctgaacgtctggcgaagtacaaccagctcatgaggattgaagaggagttgggggaggaggctcgcttcgcgggacacaacttccggaatcccagtgtgctgtgaccccctgcttgcctgaacaccggaacatcatctcattctcctggagcctctttcttgctgtcccgacccgccatagttaccttgataccttgagccccaagtcacccagaacacctcgactcacctgctctggctgttcttggcttccacaaccccttgctgtctcctactcttcctcctctctgggccccatttttggggggattccagtctgcccactttcccttctattctctctaatcttaaaaaaaaaaaaaaaatgacgactagaagaagcggtccacagaagaaccgccagcgtccgagaggagcttcaggattggtgtgttggggcgtttaaagtggggccacgtggcacgtgtgcttccctgccatccatggtgtgttaagccttgaactatgcacagagctggtgtttggggagtgctggatgtgtgtgtgttcacatttgtttgtttgtttatttatttattcacttatttatttctcagcctgtcagtcgtctgccattactcttacagtctgaaagcatcagtattttcacgtggttccatttcaagatgacctaggatgggaggttttgttagcatgggaaaggtcacagaaaggttagcaatggtttttcatttggtgcactaactgaagctcggtactttacagaatggggctgtgtacccggggacttttctcctataactctctcccccagccctaggttcctcagtctttttctccggctgcaccagagcgctgcctcattcccccgtgccatgtcccacagttgccactgtctctgtggctttgaaatgaccaccactattaaagtctgaaccacagtgcacacc

SEQ ID NO: 4 - DNA, 1162 bp, homo sapiens
agtaaaggtgatggcaggaaggcagcccccggaggcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctggggccagggaaaagatcctgcactggggatctgaagctggggagaacaggacacggggtggagaggcgaaaggagggcagagtgaagcagagagactgaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggagacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggtgagg

SEQ ID NO: 5 - DNA, 2273 bp, Chicken
acatccttctctgcagctccgtcctctctcctctctaccagcactttctcagtctctcgttcacccctgctctaggaagagtcaccatggcagttgagaggatccatgcccgagagatcctggattctcgtgggaaccccactgtagaggtggacctgtacacacacaaaggcatgtttcgagcagcggtccccagcggagcatccactggcatctatgaagcattggagctacgagataatgacaagtcacgtttcctcggaaaaggggtcctgcaggccgtggatcatatcaacagcactgtcgccccagctatcgtgggctctggcctctctgttgtagaccaagagaagatagacaatctgatgcttgagatggacggcacagagaacaaatccaagtttggtgccaatgccattctgggagtttcactggctgtgtgcaaggcaggagctgcagagaaggatgtccccctgtaccggcacattgctgacctggcaggcaactccgatctcatccttcctgtgccagctttcaatgtgatcaatggaggttcccatgcaggcaacaaattggcaatgcaggagttcatgatcctacctgtgggagctgaaagcttccgcgatgccatgcgcattggagctgaagtctatcacaatctcaagagtgttatcaaggagaagtatggcaaggatgctactaatgtgggtgacgagggaggatttgctcctaacatcctggaaaacagtgaagctctggagctcctcaaggaagctattgacaaggctggctacacagacaagatcgttatcggtatggatgtggcagcctctgagttctatcgagatggcaaatatgaccttgacttcaagtccccagatgacccaagccgctacatttctgcagatgagttgggtgacctctatcaaagctttgtacgtgcttatccagtgctttccattgaggatccctttgaccaagatgactgggaggcctggtccaagttcacagccaacgttgggattcagatcgtgggagacgacctgacagtgacaaaccccaagcgcattgagcgagctgttgaagagaaggcatgcaactgcctcctgctcaaagtcaaccagattggatctgtcacggaggccatccaagcctgtaagttggcccaggagaatggctggggtgtgatggtgagtcaccgatctggggagaccgaagacactttcattgctgatctggtcgtagcactgtgcactgggcagataaagactggtgccccctgcaggtctgaacgcctggctaaatacaaccagctcatgaggattgaggaggagcttggcgatgaagcacgctttgctggacacaactttcgcaacccaagtgttctttgaacgttgttccccaggcatagacacccttcagctctttcccacatcacagactctgaaccgtccttctcttcactactcctttttttgctctgtctcccttctctgccacctggtttcctctcacctcaaagccccacgagttcaggtatccctggctagatatacccaggtgaagggtgaaaaagaggtctgcaccctgtcccttgctttgggatcataagcatttctggatttaggcattttgtgtcttttgttcttgtgtgcatctgaatcactctgttcacatattagactatgggagcatgctgcccagcgtgtttacgttgtatgccgaagcatgtgcagtgaacatatgtgcgtcggctggtgtgtttggacaagtgtcgcacttgtagctgtgtttgaacgagtgtcaccagtggctcgctctcgttgtgcgtgtgtgtaggtgtatgtgtgctggcctacagcattgttgtgcccgggtgcatttgtgctgagccgtggtgggacctaagcgtctcttccttccgttctgagcactgttccgtgctgagctgtgcttgtcagcagcttagtgttgagtgaagaattgcttgtcactggccgtgtgtacgtgtgctgtcagtctctgggttaagcagctctgtgggatcttgtgggtcagcacatagtactgtggggcaatatattaaaacacagtcgtaaaaacacacatgctctcatttgcttatcttgaccttcttctctgtttccatccctcttgcactccttcactctctcttctgcccataatgtctgcttcaccttctagacagtcaacgctcagcaaagcaacaaactacagaaccctaaacttaattaaaatgtatttcacacctg

SEQ ID NO: 6 - DNA, 2222 bp, Rattus Sp.
cgccgccgccgtcaccaccgccaccgccaccggctgagtctgcagtcctcgaggagatcccagccatcatgtctatacagaagatctgggcccgagagatcttggactcccgtgggaatcccaccgtggaggtggatctccatactgccaaaggtcttttccgggctgcagtccccagtggggcctccactggcatctatgaggccctggagctaagggatggggacaaacagcgttacttaggcaaaggtgtcctgaaggctgtggaccacatcaacagcaccatcgcaccggccctcatcagctcaggcctctctgtggtggagcaggagaagctggacaacctgatgctggagttggatgggactgagaacaaatccaagtttggggccaatgccatcctgggtgtgtccctggccgtgtgcaaggctggggcagccgagaaggacttgcccctctatcgccacattgctcaactggctgggaactccgacctcatcctgcccgtgccggcctttaatgtgatcaacggtggctctcatgctgggaacaagttggccatgcaggagttcatgatcctcccagtgggtgctgagagctttcgggatgccatgcgacttggggccgaggtgtaccacacactcaagggggtcatcaaggacaagtacggcaaggatgccactaatgtgggggatgaaggcggctttgcccccaatatcctggagaacagcgaagctttggagctggtgaaggaagccattgacaaggctggctacacggaaaagatggtgattggtatggatgtggctgcctctgagttttaccgcgatggcaaatacgacttggatttcaagtctcccgctgacccttcccgatgcatcactggggaccagcttggggcactctaccaggactttgtccggaactatcctgtggtctccattgaagacccattcgaccaggatgactgggcagcttggtccaagttcacagccaatgtcggcatccagatagtgggtgatgacctgacggtgaccaaccccaagcgcatcgagcgggcagtggaggagaaggcctgcaactgtttgctgctcaaggtcaaccagatcggctcagtcacagaagccatccaagcgtgcaagctggcccaggagaacggctggggggttatggtgagtcatcgctctggagaaaccgaggacacgttcattgcagacctcgtagtgggactgtgtacaggtcagatcaagactggtgccccatgcagatctgaacgtctggcgaagtacaaccagctcatgaggattgaagaggagttgggggaggaggctcgcttcgcgggacacaacttccggaatcccagtgtgctgtgaccccctgcttgcctgaacaccggaacatcatctcattctcctggagcctctttcttgctgtcccgacccgccatagttaccttgataccttgagccccaagtcacccagaacacctcgactcacctgctctggctgttcttggcttccacaaccccttgctgtctcctactcttcctcctctctgggccccatttttggggggattccagtctgcccactttcccttctattctctctaatcttaaaaaaaaaaaaaaaaaaatgacgactagaagaagcgggtccacagaagaaccgccagcgtccgacaggagcttcaggattggtgtgttggggcgtttaaagtggggccacgtggcacgtgtgcttccctgccatccatggtgtgttaagccttgaactatgcacagagctggtgtttggggagtgctggatgtgtgtgtgttcacatttgtttgtttgtttatttatttattcacttatttatttctcagcctgtcagtcgtctgccattactcttacagtctgaaagcatcagtattttcacgtggttccatttcaagatgacctaggatgggaggttttgttagcatgggaaaggtcacagaaaggttagcaatggtttttcatttggtgcactaactgaagctcggtactttacagaatggggctgtgtacccggggacttttctcctataactctctcccccagccctaggttcctcagtctttttctccggctgcaccagagcgctgcctcattcccccgtgccatgtcccacagttgccactgtctctgtggctttgaaatgaccaccactattaaagtctgaatcacagtgcacacc

SEQ ID NO: 7 - protein, 28 aa, Rattus Sp.
MSIQKIWAREILDSRGNPTVEVDLHTAK

SEQ ID NO: 8 - DNA, 124 bp, Rattus Sp.
ccacccaaggagatcccagccatcatgtctatacagaagatctgggcccgagagatcttggactcccgtgggaatcccaccgtggaggtggatctccatactgccaaaggtgatgattagcgtg

SEQ ID NO: 9 - DNA, 1162 bp, Rattus Sp.; identical to SEQ ID NO: 4

SEQ ID NO: 10 - protein, 28 aa, Rattus Sp.
MSIQKIWAREILDSRGNPTVEVDLHTAK

SEQ ID NO: 11 - DNA, 124 bp, Rattus Sp.; identical to SEQ ID NO: 8

SEQ ID NO: 12 - DNA, 513 bp, Rattus Sp.
gtgaggcccgtatcgggccgcatccccttactctgtccttgccgcgtccactccccgttatcctgggcttagcgcccccgggtgtgtgtgtgtgtgtgtgtgtgtgtgtgtgtgtgtgtgtgtgtgtgtgtgtgtgtgtgtgtgtgacgctgcgtacctgtcacctgccttccctggcaccctccatccccggccttcgcggacagctcccggctttctggctgtcacctcctcctggtttaggtaccagcttttctctctctctctctctctctctctctctctctctctctctctctctctctctctccctgcacccgcttctctaactccacggttccaccctcgcctgcccctctccccatgcctgtattcgtccagggtccggccgatttgtttcttaagttcgatgtctccgcaggcggcgcgtgttttcctggcacgttcatttttgctttgcgtttgcgaagataccctctcatccttccttggctgcttcagacttgctctaga

SEQ ID NO: 13 - protein, 434 aa, Rattus Sp.
MSIQKIWAREILDSRGNPTVEVDLHTAKGLFRAAVPSGASTGIYEALELRDGDKQRYLGKGVLKAVDHINSTIAPALISSGLSVVEQEKLDNLMLELDGTENKSKFGANAILGVSLAVCKAGAAEKDLPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKMVIGMDVAASEFYRDGKYDLDFKSPADPSRCITGDQLGALYQDFVRNYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGEEARFAGHNFRNPSVL

SEQ ID NO: 14 - DNA, 362 bp, Rattus Sp.
gcgaccccttctacatgtgtctagattgcagcctgcccctccccactccctacccactgcagtagacctcttccccagccctcccgcctctggccccgcccactggcctcaggctccacccttctaagcctcttatctgtctccttccctcttgtccacccaaggagatcccagccatcatgtctatacagaagatctgggcccgagagatcttggactcccgtgggaatcccaccgtggaggtggatctccatactgccaaaggtgatgattagcgtggactgtctgtagctcctacaacccctacgccctaggtctggaaagggttgaggtcttccttcaaatccaggcatggggtggct

SEQ ID NO: 15 - protein, 434 aa, Rattus Sp.; identical to SEQ ID NO: 13

SEQ ID NO: 16 - DNA, 2284 bp, Rattus Sp.
gccgccccctgcccaccccccgcccgcgctgggagccgcagccgccgccactcctgctctctctgcgccgccgccgtcaccaccgccaccgccaccggctgagtctgcagtcctcgaggagatcccagccatcatgtctatacagaagatctgggcccgagagatcttggactcccgtgggaatcccaccgtggaggtggatctccatactgccaaaggtcttttccgggctgcagtccccagtggggcctccactggcatctatgaggccctggagctaagggatggggacaaacagcgttacttaggcaaaggtgtcctgaaggctgtggaccacatcaacagcaccatcgcaccggccctcatcagctcaggcctctctgtggtggagcaggagaagctggacaacctgatgctggagttggatgggactgagaacaaatccaagtttggggccaatgccatcctgggtgtgtccctggccgtgtgcaaggctggggcagccgagaaggacttgcccctctatcgccacattgctcaactggctgggaactccgacctcatcctgcccgtgccggcctttaatgtgatcaacggtggctctcatgctgggaacaagttggccatgcaggagttcatgatcctcccagtgggtgctgagagctttcgggatgccatgcgacttggggccgaggtgtaccacacactcaagggggtcatcaaggacaagtacggcaaggatgccactaatgtgggggatgaaggcggctttgcccccaatatcctggagaacagcgaagctttggagctggtgaaggaagccattgacaaggctggctacacggaaaagatggtgattggtatggatgtggctgcctctgagttttaccgcgatggcaaatacgacttggatttcaagtctcctgctgacccttcccgatgcatcactggggaccagcttggggcactctaccaggactttgtccggaactatcctgtggtctccattgaagacccattcgaccaggatgactgggcagcttggtccaagttcacagccaatgtcggcatccagatagtgggtgatgacctgacggtgaccaaccccaagcgcatcgagcgggcagtggaggagaaggcctgcaactgtttgctgctcaaggtcaaccagatcggctcagtcacagaagccatccaagcgtgcaagctggcccaggagaacggctggggggttatggtgagtcatcgctctggagaaaccgaggacacgttcattgcagacctcgtagtgggactgtgtacaggtcagatcaagactggtgccccatgcagatctgaacgtctggcgaagtacaaccagctcatgaggattgaagaggagttgggggaggaggctcgcttcgcgggacacaacttccggaatcccagtgtgctgtgaccccctgcttgcctgaacaccggaacatcatctcattctcctggagcctctttcttgctgtcccgacccgccatagttaccttgataccttgagccccaagtcacccagaacacctcgactcacctgctctggctgttcttggcttccacaaccccttgctgtctcctactcttcctcctctctgggccccatttttggggggattccagtatgcccactttcccttctattctctctaatcttaaaaaaaaaaaaaaaatgacgactagaagaagcggtccacagaagaaccgccagcgtccgacaggagcttcaggattggtgtgttggggcgtttaaagtggggccacgtggcacgtgtgcttccctgccatccatggtgtgttaagccttgaactatgcacagagctggtgtttggggagtgctggatgtgtgtgtgttcacatttgtttgtttgtttatttatttattcacttatttatttctcagcctgtcagtcgtctgccattactcttacagtctgaaagcatcagtattttcacgtggttccatttcaagatgacctaggatgggaggttttgttagcatgggaaaggtcacagaaaggttagcaatggtttttcatttggtgcactaactgaagctcggtactttacagaatggggctgtgtacccggggacttttctcctataactctctcccccagccctaggttcctcagtctttttctccggctgcaccagagcgctgcctcattcccccgtgccatgtcccacagttgccactgtctctgtggctttgaaatgaccaccactattaaagtctgaaccacagtgcacacca

SEQ ID NO: 17 - DNA, 2715 bp, Rattus Sp.
cggggatctctgagtttgaggccagcctggtctgcagagtgattttcaggacagccagggctacacagtagaagtctgtcttggaaggaaggaaggaaggagagaaagaaggaaggaaggaaggagagaaggaaggaaggaaggaaaacaacttcaaggccagatgctgtagcatatgcctatagtctagggcttggaagtcaacggcggcaggagaattgatgcaagtgtgagggagaagccagatccatacggtgcgtagttctgtgccagccaggggtacacagtgggattcagtcaacagcactaacagtaacaaaaaaaccagatgtgtggctcatacctataattccagcactcaggaacccgaggcaggaggatttctgtgagattgaggccagcctggactgcagagccagactctgtcacacccctcctccacacacacacacacacacacacacacacacacacacacacacacacacaaagatacacacacacagagacacacacacacagagacacacacacagagacacacacgtgcgcgcacgcacacacacaacatcacaacatgcacacacgtgctcgcacacacgcgcgcgcacgcacacacacacggaaggaggaaggaggaaggaggagatggatgaacacatgcagggtcagagggatttttctctaagctcctcttttctgatggacaattctttcaattttttttgtggttctatcaatcaaacccagggctgtgtgcatgtgaaatctgtaccccatcactgagcccaacacaacgccagtagagctcagagtgaggagtgctccttgaggcccagggcacctatggagatcagtctgcaatacttaacactggattcataaatgttcgaaaccacagagttttggaaagaagaacattacaagactgagctttttattcaagctggggggctcaatccatccttagctctgggttccttactgaaggaagcactcccaccccacagtaccccactcttagctctgagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctggggccagggaaaagatcctgcactggggatctgaagctggggagaacaggacacggggtggagaggcgaaaggagggcagagtgaagcagagagactgaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggagacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggc

SEQ ID NO: 18 - DNA, 4144 bp, Rattus Sp.
gaattctactcttgtctccctaggtagcctctctccacagaccctattcacacaccccctccaatagtccccctccttccactctggcttttttcacccgccttccttcacacctatctctcctacttaaaatctcactggatctgtctttagctttggagctggtgaaggaagccattgacaaggctggctacacggaaaagatggtgattggtatggatgtggctgcctctgagttttaccgcgatggcaaatacgacttggatttcaagtctcctgctgacccttcccgatgcatcactggggaccagcttggggcactctaccaggactttgtccggaactatcctggtgagagtgagggattctgggagataggaaagggaaagaacggagataggcagaggtgaactttatggggtgcagagtctggggcctggtgacgtcctgaagcagggagaagaggcagtaggttcttacaatgcagacctttgagactgtgcatgcaactgtgactgggctcctccgcctcgtttcagaccctgggctctgagagctacttgagtcctagggaaagcactggaaggagtcggggatgggaagaacatgagtggaggtgacagaaaagtgccaggctttagtatccttgattgatgaagatagatactgaatggagtcagcgcggctgcctggggcggggcagggaggggatcactgaggtacagagtctccacagctacaaagggcctccatcctgcttcgtggaggcagttggcctccatcatagcaggcttctctgggaagaaccagccccctctccacaccctctgcagtggtctccattgaagacccattcgaccaggatgactgggcagcttggtccaagttcacagccaatgtcggcatccagatagtgggtgatgacctgacggtgaccaaccccaagcgcatcgagcgggcagtggaggagaaggcctgcaactgtttgctgctcaaggtcaaccagatcggctcagtcacagaagccatccaagcgtgagtggcttctggcctcctcccaccttgtgtcttcaggactcctcttgagagccccccctagctggattctgccctggaactctggtgaacactggctacccttcacactgttctcacttctagtctcttcattcttcacgatctgccctcacgcccctaagtcaccttccaccaggtgcattcctgacatagatcaagcccggggctgggagggaagagctgtgcttcagggcaggtcagtgctggtgtctgctccaggtgcaagctggcccaggagaacggctggggggttatggtgagtcatcgctctggagaaaccgaggacacgttcattgcagacctcgtagtgggactgtgtacaggtcaggtgagtagagccagtccgagggatcaggagagatctttgtggggttagtgggttctgggcccaggtagttactgtcctgccaaaaaaaaatctgagtcagaaaagtttcaagagtagaagtctggccaggcgtagtggtgcacacctttaatcccagcactcaggaggcagaggcaggtggatctctgtgagtttaaggccagtttggtttatatagtgagtttcaggacagtcagggctacactgagggactccttaacaaacgaagacgtgaaaagagtagaagttccgagctggtgagatggcccaggaggttcaagtgcccggtgctaagcctgactgctagcttcagtttgattcatgggacccacaggttagaaggagagaaccaactcccaccagttgtcctctgaccttccaatgcacaccaggcaagagcagacccacagacatgcatacagacatgaaataaagtattaatttttataattaattaattaataattaatagatacaaatgcagatgtttcagggctggaggcttagctcagttgcaggtacttcccacatgcgcaagaccctgggttccattaaaaaaaggggaggggggaggtagggtatgacctgcatgcctttagtcctagcactcaggagacagaggaaggaggatttctatgaattcaccagcctagtctacttagcgagttctaggacggctatagtaaaattctgtcccacaaaaacaaaaaatagacctggacttggggtcacatcctttagccccaacacttcgggaagtagaagcaggaggatcaggagttcagggctaacctgtcccatttgaggccaactttcataaaattgtctcaaaataaaaaaaagtgtgtgtgtgtgtgtgtgtgtgtgtgtgtgtgtgtgtgtgtgtgtgttgtgttgggggtaaaggggagcaaggttgttctatcaggtgaaggagatctttaaacggcatatcttctggaatgtttcagatcaagactggtgccccatgcagatctgaacgtctggcgaagtacaaccagctcatgaggtgaggagggtccctgaggaacaagaacccgagacccagagctgaacaccctattaggccaccattccatctcttgcatccaggagctggatagtactggctgccaagaaaatggttttgtttagtgggttcaagggtggtgtgactgacagatcactaacgtacctgaaaaccagggaatgttcaccttggggcagtcctcaagggaggagcctggcgggatccagagagaaaagcagatctcctctcccctccctccccctccccatgctccattccggttcatcgtcaggattgaagaggagttgggggaggaggctcgcttcgcgggacacaacttccggaatcccagtgtgctgtgaccccctgcttgcctgaacaccggaacatcatctcattctcctggagcctctttcttgctgtcccgacccgccatagttaccttgataccttgagccccaagtcacccagaacacctcgactcacctgctctggctgttcttggcttccacaaccccttgctgtctcctactcttcctcctctctgggccccatttttggggggattccagtatgcccactttcccttctattctctctaatcttaaaaaaaaaaaaaaaatgacgactagaagaagcggtccacagaagaaccgccagcgtccgacaggagcttcaggattggtgtgttggggcgtttaaagtggggccacgtggcacgtgtgcttccctgccatccatggtgtgttaagccttgaactatgcacagagctggtgtttggggagtgctggatgtgtgtgtgttcacatttgtttgtttgtttatttatttattcacttatttatttctcagcctgtcagtcgtctgccattactcttacagtctgaaagcatcagtattttcacgtggttccatttcaagatgacctaggatgggaggttttgttagcatgggaaaggtcacagaaaggttagcaatggtttttcatttggtgcactaactgaagctcggtactttacagaatggggctgtgtacccggggacttttctcctataactctctcccccagccctaggttcctcagtctttttctccggctgcaccagagcgctgcctcattcccccgtgccatgtcccacagttgccactgtctctgtggctttgaaatgaccaccactattaaagtctgaaccacagtgcacaccaccccttgtctgaggacttttactctttgctccatatgagaggaggaagagaaaggactggatattggtgaacctagagagcagatctggggagggcaagtcactgaactcatgaccaagtttgcagcattgctggctatgtggaacccatggtaaaaaatcccccaaagacagggtttctctgtgaagccctggctgtcctggagctcaccctgtagaccatgctggccccaaacctatagtgattagcctgcctctgcctctgggtgctgggattaaaattgtgtactgccacacctgaccaaactaaggactcttaacctgtgatacaggagtccctcaaacatcagggattgagtgtatctggagtgaggaataaggcatttctggttcccaaagtctttgagggaagaagggataaagtggtagactagcattcctgtacgggtcctcaacagaattc

SEQ ID NO: 19 - DNA, 1980 bp, Rattus Sp.
ggccccggagtgtcctcccctttcctcgtcgcgtgtctttttccgaggtcttttccgggctgcagtccccagtggggcctccactggcatctatgaggccctggagctaagggatggggacaaacagcgttacttaggcaaaggtgatgactctcctctcccagcaggccccagtcccctagatgccttgccctgcctctcctcagtctccaccccctctcctggcctgagcagcccctttccctttctctaggtgtcctgaaggctgtggaccacatcaacagcaccatcgcaccggccctcatcagctcagtgaggcccgctctttcctgggggtggctgctaggccagaatcttacaagaaaccttgggtccagggaggaacgaggggaggcaaaaacaaacaaacaaacaaacaaacaactccacacacacacacaccccttccctccccaccccccccccactctttaggaagttgtggttacaaatgcaagggttgggaaaatacttcttactgcccctgttgaaattcaagatcccaaaagctggtagggcagggatctggcagtgcctgctcagacataaggctttgtgtgaatggaccctcagtcttaggctccagcctccatcttgactctgggtaatgggggctctgcacagggcctctctgtggtggagcaggagaagctggacaacctgatgctggagttggatgggactgagaacaaatgtaagtagggagggcaggtggaatagctaagcacagagaactgaggagccatctggggacacttggactgtcagagaacctccttctccctcacccaaagccactgtagtgtgtgtgtgtgtgtgtgtgtgtgtgtgtgtgtgtgtgtgtgacactagacaggaggaaaaacttccctaaagcaggatgacaggggctggcatggggtagtctgggggatgcttaggaagaggtgaggccttcccttacttgtcatctctgtccccaaccccactgcttttgggaggtgcagggtggggagcctcctttactggatttttctggggagtataggtaggggcaggggctacaagagagggggacatttttactcggcgccttcctctgtgatctctcagccaagtttggggccaatgccatcctgggtgtgtccctggccgtgtgcaaggctggggcagccgagaaggacttgcccctctatcgccacattgctcaactggctgggaactccgacctcatcctgcccgtgccggtgagcattgcccttgcctagcccttccaggggaggagggagaatgcaaccaagtgagggatgaagggaggaaggcgaacagacagcagctaaagagaaaggacgggatgtgagaactacctggcggctggggatctcaggttggcattctcggggcaggaggaaaggctctgactcgtctgaccatctgtggctccccaggcctttaatgtgatcaacggtggctctcatgctgggaacaagttggccatgcaggagttcatgatcctcccagtgggtgctgagagctttcgggatgccatgcgacttggggccgaggtgtaccacacactcaagggggtcatcaaggacaagtacggcaaggatgccactaatgtgggggatgaaggcggctttgcccccaatatcctggagaacagcgaaggtgaggcccggagcccttaccccagctgaatctcccgttaggaaagctggcacacgcgggtttttgaacttctgaaaatctcaaatggtagtagcccctagggtgagctcccggggccttcctatttcccacatagaccctggagctcagtctgtgacctgctgagctaaacctgttgcataggtcctcaccagactgctgagaatattggtaagtggtctgggtacggtccatttcatcctttaaggacacagtctcagttagatgacatattctatgctccaggggggt

SEQ ID NO: 20 - DNA, 1316 bp, Rattus Sp.
ctgcagggacagtaaaggtgatggcaggaaggcagcccccggaggcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctggggccagggaaaagatcctgcactggggatctgaagctggggagaacaggacacggggtggagaggcgaaaggagggcagagtgaagcagagagactgaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggagacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggtgaggcccgtatcggcctccccttactctgtccttgccgcgtccactccccgttatctgggcttagcgcccccgggtgtgtgtgtgtgtgtgtgtgtgtgtgtgtgtgtgtgtgtgtgtgtgtgtgtgtgtgtgtgtgtgtcgtccgcg

SEQ ID NO: 21 - protein, 434 aa, Homo Sapiens
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 22 - protein, 434 aa, Rattus Sp.; identical to SEQ ID NO: 13

SEQ ID NO: 23 - protein, 434 aa, homo sapiens; identical to SEQ ID NO: 21

SEQ ID NO: 24 - protein, 434 aa, Chicken
MAVERIHAREILDSRGNPTVEVDLYTHKGMFRAAVPSGASTGIYEALELRDNDKSRFLGKGVLQAVDHINSTVAPAIVGSGLSVVDQEKIDNLMLEMDGTENKSKFGANAILGVSLAVCKAGAAEKDVPLYRHIADLAGNSDLILPVPAFNVINGGSHAGNKLAMQEFMILPVGAESFRDAMRIGAEVYHNLKSVIKEKYGKDATNVGDEGGFAPNILENSEALELLKEAIDKAGYTDKIVIGMDVAASEFYRDGKYDLDFKSPDDPSRYISADELGDLYQSFVRAYPVLSIEDPFDQDDWEAWSKFTANVGIQIVGDDLTVTNPKRIERAVEEKACNCLLLKVNQIGSVTEAIQACKLAQENGWGVMVSHRSGETEDTFIADLVVALCTGQIKTGAPCRSERLAKYNQLMRIEEELGDEARFAGHNFRNPSVL

(19) United States
(12) Patent Application Publication
(10) Pub. No.: US 2005/0142616 A1
(43) Pub. Date: Jun. 30, 2005

(54) Diagnostic test for neuropsychiatric systemic lupus erythematosus

(76) Inventors: Veneta Hanson, Bronxville, NY (US); Harry Spiera, New York, NY (US)

Correspondence Address: John P. White, Esq.;Cooper & Dunham LLP, 1185 Avenue of the Americas, New York, NY, 10036, US

(21) Appl. No.: 10/676,691

(22) Filed: Sep. 30, 2003

(51) Int. Cl. G01N 33/53; G01N 33/567; C07K 16/42

(52) U.S. Cl. 435/7.2; 435/7.5; 530/387.2

(57) ABSTRACT

This invention provides methods for assessing the likelihood that a patient is suffering neuropsychiatric systemic lupus erythematosus by detecting autoantibodies in a sample from the patient. This invention also provides diagnostic kits for the same.

The invention disclosed herein was made with Government support under a grant from the National Institute for Arthritis and Musculoskeletal Diseases (NIAMD) No. AR42559. Accordingly, the U.S. Government has certain rights in this invention.

Throughout this application, various publications are referenced in parentheses by number. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Neurological, psychiatric and behavioral abnormalities are reported in up to 70% of systemic lupus erythematosus (SLE) patients (1), a condition referred to as SLE with neuropsychiatric disease, or neuropsychiatric systemic lupus erythematosus (NPSLE). Neurological syndromes may involve the central, peripheral and autonomic nervous system, while psychosis is the most prevalent psychiatric presentation, followed by cognitive dysfunction, mood changes and acute confusional states (2). Assessment of central nervous system (CNS) involvement with SLE is not a precise process and involves serologic studies of autoantibodies, primarily to dsDNA, the small ribonucleoprotein and ribonucleoprotein antigens, and to phospholipid. Although helpful in the diagnosis of lupus, the presence of these autoantibodies does not correlate well with neuropsychiatric disease. Some correlation of serum autoantibodies to neuronal antigens and to the ribosomal P protein with neuropsychiatric manifestations exist in lupus, and in some of the reports these autoantibodies are implicated in the pathogenic mechanisms of the disease (3,4,5). Characterization of precise autoantibody specificities in the brain and the evaluation of these autoantibodies as markers for neuropsychiatric disease would provide a better serological tool for the diagnosis of NPSLE.

We have previously discovered the presence of autoantibodies to a synaptic membrane protein in patients with NPSLE (6). Here we further characterize the target antigen, and evaluate association of the respective autoantibodies with specific clinical presentations of NPSLE. Surprisingly, the antigen shows high homology to the glycolytic enzyme gamma enolase. These characteristics permit using the subject's autoantibodies as markers for neuropsychiatric disease in the diagnosis of NPSLE and other autoimmune neuropsychiatric disorders, which in turn permits individualized therapy to treat the disease.

### SUMMARY OF THE INVENTION

This invention provides a method of assessing the likelihood that a patient is suffering from neuropsychiatric systemic lupus erythematosus which comprises:
a) obtaining a fluid sample from the subject;
b) contacting the sample with an agent which forms a complex with an autoantibody to a protein comprising consecutive amino acids having the sequence set forth in SEQ ID NO:21 or 23, under conditions permitting any such autoantibody present in the sample to complex with the agent; and
c) detecting the presence of any autoantibody-agent complex formed in step (b);
   wherein the detection of autoantibody-agent complex in step (c) indicates that the patient is likely suffering from neuropsychiatric systemic lupus erythematosus.

This invention also provides method of assessing the likelihood that a patient is suffering from neuropsychiatric systemic lupus erythematosus which comprises:
a) providing a solid support to which an agent which forms a complex with an autoantibody to a protein comprising consecutive amino acids having the sequence set forth in SEQ ID NO:21 or 23, under conditions permitting any such autoantibody present in the sample to complex with the agent is bound;
b) contacting the solid support from (a) with a fluid sample from the subject;
c) removing any of the autoantibody which is not bound to the solid support; and
d) detecting the presence of autoantibody bound to the solid support,
   wherein the detection of autoantibody bound to the solid support in step (d) indicates that the patient is likely suffering from neuropsychiatric systemic lupus erythematosus.

This invention also provides a diagnostic kit which comprises a container comprising an agent which forms a complex with an autoantibody to a protein comprising consecutive amino acids having the sequence set forth in SEQ ID NO:21 or 23, which agent is labeled with a detectable marker.

This invention also provides a diagnostic kit which comprises a container comprising a solid support to which an agent which forms a complex with an autoantibody to a protein comprising consecutive amino acids having the sequence set forth in SEQ ID NO:21 or 23 is bound, which agent is labeled with a detectable marker.

### BRIEF DESCRIPTION OF THE FIGURES

FIG. 1: Western blot analysis using commercially available preparation of gamma enolase. The data show that all five patients reacted with the commercial preparation of gamma enolase as well as the 50 kD protein, see lanes A1-5 for commercially available NSE and lanes B1-5 for purified 50 kD protein. Lane 6 is control.

FIG. 2A-E: Time progression photographs of cultured B104 neuroblastoma cells in the presence of autoantibodies. Cells gradually detached from the bottom of the flask, formed free floating clumps of cells that appeared dead on day six, leaving fewer viable cells adherent to the bottom of the flask.

FIG. 3: Immunofluorescent localization experiments with affinity purified autoantibodies on cells remaining attached after day six, show vesicular aggregates in the perinuclear region and in the extracellular environment

FIG. 4A-B: mRNA sequence encoding neuron-specific enolase (SEQ ID NO:1).

FIG. 5A-F: Human DNA sequence encoding neuron-specific enolase (SEQ ID NO:2).

FIG. 6A-B: Rat completed cds mRNA sequence encoding neuron-specific enolase (SEQ ID NO:3).

FIG. 7: Neuron-specific enolase mRNA and intron (SEQ ID NO:4).

FIG. 8A-B: Chick neuron-specific enolase mRNA complete cds (SEQ ID NO:5).

FIG. 9A-B: Rat neuron-specific enolase mRNA complete cds (SEQ ID NO:6).

FIG. 10: Amino acid sequence of neuron specific enolase and encoding DNA (SEQ ID NO:7) and (SEQ ID NO:8) respectively.

FIG. 11: DNA encoding rat neuron-specific enolase—5′ end (SEQ ID NO:9).

FIG. 12: Rat neuron-specific enolase—exon 1 and protein product (SEQ ID NO:10) and (SEQ ID NO:11) respectively.

FIG. 13: Rat, neuron cells PC12 DNA encoding rat neuron-specific enolase—intron 1 (SEQ ID NO:12).

FIG. 14: R. norvegicus gene encoding neuron-specific enolase, exon 2 and joined CDS; protein sequence and DNA sequence (SEQ ID NO:13) and (SEQ ID NO:14) respectively.

FIG. 15: R. norvegicus enolase 2, gamma (Eno2) (SEQ ID NO:15).

FIG. 16A-B: R. norvegicus enolase 2, gamma (Eno2) mRNA (SEQ ID NO:16).

FIG. 17A-B: R. norvegicus neuron-specific enolase gene, 5′ flanking region (SEQ ID NO:17).

FIG. 18A-C: R. norvegicus gene encoding neuron-specific enolase gene, exons 8-12 (SEQ ID NO:18).

FIG. 19A-B: R. norvegicus gene encoding neuron-specific enolase gene, exons 3-7 (SEQ ID NO:19).

FIG. 20: R. norvegicus gene encoding neuron-specific enolase gene, exon 1 (SEQ ID NO:20).

FIG. 21: Homo Sapiens neuron-specific enolase (SEQ ID NO:21).

FIG. 22: Rat neuron-specific enolase (SEQ ID NO:22).

FIG. 23: Human neuron-specific enolase (SEQ ID NO:23).

FIG. 24: Chick neuron-specific enolase (SEQ ID NO:24).

### DETAILED DESCRIPTION OF THE INVENTION

Definitions

As used herein, and unless stated otherwise, each of the following terms shall have the definition set forth below.

“Antibody” shall include, by way of example, naturally occurring antibodies. The term “Autoantibody” is applied to antibodies produced by the subject and directed against naturally occurring components of the same subject. “Autoantibody-agent complex” means an association between the agent and the autoantibody, for example that between an epitope and a paratope.

“Autoimmune”, when applied to a disease, shall mean a state where the etiology of the disease is the subject's own immune system. By way of example, this includes diseases caused by inappropriately produced autoantibodies, or caused by autoantibodies or lymphocytes that attack molecules, cells, or tissues of the organism producing them.

“Immunogenic” when applied to an agent shall mean an agent that elicits an immune response in a patient.

“Neuropsychiatric” refers to psychiatric diseases or conditions with an underlying neurological basis.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Operably affixed” in reference to an antigen protein shall mean that the protein is affixed to a molecule or surface in such a way as to permit any antibodies directed towards an epitope present on the protein to form a complex with the protein.

Conditions permitting autoantibodies to complex with agents, e.g. proteins, means conditions which allow an antibody or autoantibody to complex with, or bind to, the agent/protein. Conditions allowing specific interaction mean conditions permitting the antibody or autoantibody to bind to a detectably greater degree (e.g., at least 2-fold over background) than the antibody binds to substantially all other epitopes in a reaction mixture comprising the particular epitope. Such ‘immunologically reactive’ conditions are dependent upon the format of the antibody binding reaction and typically are those utilized in immunoassay protocols. See Harlow and Lane, Antibodies, A Laboratory Manual, Cold Spring Harbor Publications, New York (1988), for a description of immunoassay formats and conditions.

A variety of immunoassay formats may be used to detect antibodies reactive with a particular agent. For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective reactivity.

### Embodiments of the Invention

This invention provides a method of assessing the likelihood that a patient is suffering from neuropsychiatric systemic lupus erythematosus which comprises:
a) obtaining a fluid sample from the subject;
b) contacting the sample with an agent which forms a complex with an autoantibody to a protein comprising consecutive amino acids having the sequence set forth in SEQ ID NO:21 or 23, under conditions permitting any such autoantibody present in the sample to complex with the agent; and
c) detecting the presence of any autoantibody-agent complex formed in step (b);
   wherein the detection of autoantibody-agent complex in step (c) indicates that the patient is likely suffering from neuropsychiatric systemic lupus erythematosus.

This invention further provides the instant method, wherein the fluid sample comprises sera, plasma, urine, saliva, synovial fluid, cerebro-spinal fluid, or lymph.

This invention further provides the instant method, wherein the agent is an antibody or fragment thereof which binds to the autoantibody and is labeled with a detectable marker.

This invention further provides the instant method, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

This invention further provides the instant method, wherein the protein or the immunogenic fragment thereof is labeled with a detectable marker.

This invention further provides the instant method, wherein the detectable marker is a radioisotope, a chromophore, a biomolecule, a fluorophore, a radiolabeled molecule, a dye, an affinity label, an antibody, biotin, streptavidin, a metabolite, a mass tag, or a dextran.

This invention further provides the instant method, wherein the detecting in step (c) comprises contacting the autoantibody-agent complex with a second antibody which binds to the autoantibody-agent complex and is labeled with a detectable marker.

This invention further provides the instant method, further comprising determining the amount of complex formed in step (b) and comparing such amount with a standard, wherein a greater amount of complex formed in step (b) than in the standard indicates that the subject is likely suffering from neuropsychiatric systemic lupus erythematosus.

This invention further provides the instant method, wherein the standard is a fluid sample comprising sera, plasma, urine, saliva, synovial fluid, cerebro-spinal fluid, or lymph from a patient not suffering from neuropsychiatric systemic lupus erythematosus.

This invention also provides method of assessing the likelihood that a patient is suffering from neuropsychiatric systemic lupus erythematosus which comprises:
a) providing a solid support to which an agent which forms a complex with an autoantibody to a protein comprising consecutive amino acids having the sequence set forth in SEQ ID NO:21 or 23, under conditions permitting any such autoantibody present in the sample to complex with the agent is bound;
b) contacting the solid support from (a) with a fluid sample from the subject;
c) removing any of the autoantibody which is not bound to the solid support; and
d) detecting the presence of autoantibody bound to the solid support,
   wherein the detection of autoantibody bound to the solid support in step (d) indicates that the patient is likely suffering from neuropsychiatric systemic lupus erythematosus.

This invention further provides the instant method, wherein the agent is a protein comprising consecutive amino acids having the sequence set forth in SEQ ID NO:21, SEQ ID NO:22, SEQ ID NO:23, SEQ ID NO:24, or an immunogenic fragment thereof and wherein the detecting of the presence of autoantibody bound to the solid support with an antibody which binds to the autoantibody is labeled with a detectable marker.

This invention further provides the instant method, wherein the fluid sample comprises sera, plasma, urine, saliva, synovial fluid, cerebro-spinal fluid, or lymph.

This invention further provides the instant method, wherein the detectable marker is a radioisotope, a chromophore, a biomolecule, a fluorophore, a radiolabeled molecule, a dye, an affinity label, an antibody, biotin, streptavidin, a metabolite, a mass tag, or a dextran.

This invention further provides the instant method, further comprising determining the amount of complex formed in step (b) and comparing such amount with a standard, wherein a greater amount of complex formed in step (b) than in the standard indicates that the subject is likely suffering from neuropsychiatric systemic lupus erythematosus.

This invention further provides the instant method, wherein the standard is a fluid sample comprising sera, plasma, urine, saliva, synovial fluid, cerebro-spinal fluid, or lymph from a patient not suffering from neuropsychiatric systemic lupus erythematosus.

This invention also provides a diagnostic kit which comprises a container comprising an agent which forms a complex with an autoantibody to a protein comprising consecutive amino acids having the sequence set forth in SEQ ID NO:21 or 23, which agent is labeled with a detectable marker.

This invention further provides the instant kit, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

This invention further provides the instant kit, wherein the agent is an antibody or fragment thereof which binds to the autoantibody and is labeled with a detectable marker.

This invention also provides a diagnostic kit which comprises a container comprising a solid support to which an agent which forms a complex with an autoantibody to a protein comprising consecutive amino acids having the sequence set forth in SEQ ID NO:21 or 23 is bound, which agent is labeled with a detectable marker.

This invention further provides the instant kit, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

This invention further provides the instant kit, wherein the agent is an antibody or fragment thereof which binds to the autoantibody and is labeled with a detectable marker.

Neuropsychiatric presentations included in the instant method include schizophrenia. Other embodiments include cognitive dysfunctions, memory disorders, complex attention disorders, psychosis, anxiety disorders and depression, acute confusional state, mood disorders, headache, seizure disorders. Other neuropsychiatric presentations include movement disorders and aseptic meningitis.

In one embodiment, the patient has been diagnosed with systemic lupus erythematosus but not NPSLE. In one embodiment the patient has NPSLE. In one embodiment the patient presents with neuropsychiatric disease symptoms. In one embodiment the patient has NPSLE wherein the neuropsychiatric component is caused by an autoimmune disease. In one embodiment the instant method is used to determine whether the neuropsychiatric component of the NPSLE is autoimmune or not. In one embodiment the method is used to determine what therapy the patient should receive for treatment of the neuropsychiatric disease, e.g. whether an autoimmune therapy should be employed.

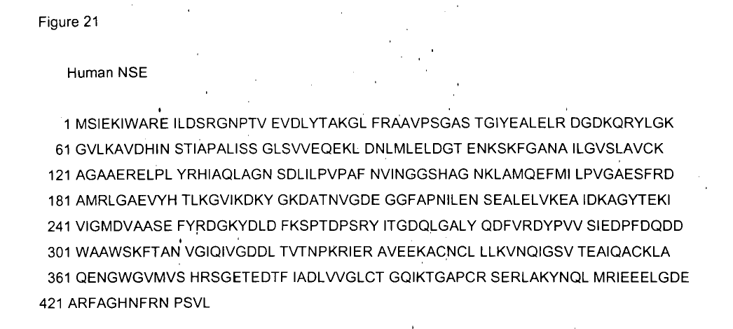

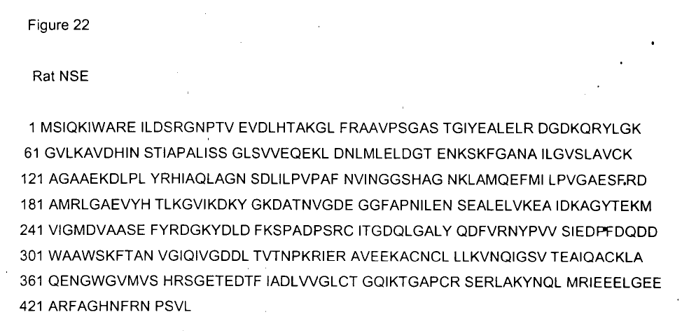

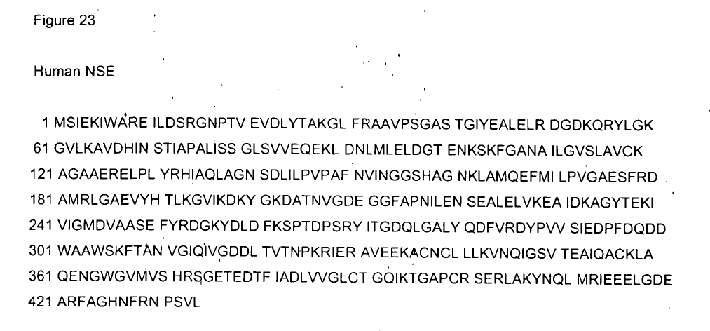

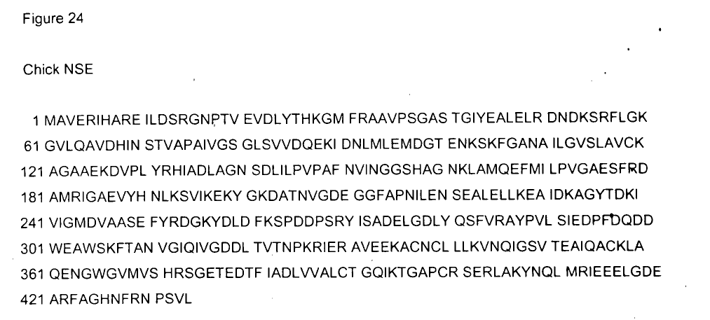

In one embodiment the agent is gamma enolase. In a further embodiment the enolase is human or bovine. In different embodiments the gamma enolase, or neuron-specific enolase, has the sequence shown in FIGS. 21-24 (SEQ ID NOs.:21-24) or is an immunogenic fragment thereof. In differing embodiments the agent is a protein encoded by a nucleic acid that encodes the proteins shown in FIGS. 21-24 (SEQ ID NOs.:21-24). In other embodiments the agent is a protein comprising consecutive amino acids comprising one or more of the sequences set forth in SEQ ID NOs.: 25-41. Acitve fragments of antibodies include F_{AB} portions.

In the described methods the agent to which the autoantibody complexes, may be labeled with a detectable marker, for example, a biomolecule, a fluorescent label, a radiolabeled molecule, a dye, a chromophore, an affinity label, an antibody, biotin, streptavidin, a metabolite, a mass tag, or a dextran.

In one embodiment the autoantibody-agent complex is detected by attaching a detectable marker to all antibodies present in the sample, including autoantibodies, and then detecting any marker present when the agent has been exposed to the sample and washed to remove any unbound sample. In another embodiment a detectable marker is attached to a secondary antibody, i.e. an antibody that will recognize the autoantibody. In another embodiment the detectable marker is attached to the agent.

This invention further provides a solid surface having an antigen protein as described hereinabove, or a fragment thereof, operably affixed thereto. This solid surface of can be, for example, glass, silica, diamond, quartz, gold, silver, metal, polypropylene, or plastic. The solid surface can be, for example, present on a bead, a chip, a wafer, a filter, a fiber, a porous media, or a column. Supports suitable for Western blot, radioimmunoassay, surface plasmon resonance chip assay, radioassay, or an enzyme-linked immunosorbent assay are encompassed.

In one embodiment the fluid sample is a sample from a patient which sample normally contains antibodies. In another embodiment the fluid sample is a sample from a patient which sample contains antibodies under pathological conditions.

This invention will be better understood by reference to the Experimental Details which follow, but those skilled in the art will readily appreciate that the specific experiments detailed are only illustrative of the invention as described more fully in the claims which follow thereafter.

Experimental Details

We previously reported the presence of autoantibodies to a 50 kD synaptic membrane protein in patients with systemic lupus erythematosus and neuropsychiatric disease (NPSLE). This study identifies and further characterizes the 50 kD synaptic membrane protein as an autoantigen in NPSLE, and explores the effects of the associated autoantibodies on neuronal cells in culture. We evaluated the presence of autoantibodies from patients with systemic lupus erythematosus and neuropsychiatric disease, as well as control groups of patients with SLE without neuropsychiatric disease, and patients with neuropsychiatric disease alone, for their reactivity with the 50 kD protein, using Western blot analysis. The identity of the 50 kD protein was determined following chromatographic purification and mass spectroscopy analysis. The effect of autoantibodies to the 50 kD protein on neuronal cultures was evaluated using affinity purified autoantibodies and B104 neuroblastoma cells. We disclose that autoantibodies to gamma enolase cocultured with neuronal cells diminish the survival of B104 neuroblastoma cells.

We also disclose here that the 50 kD synaptic membrane antigen is highly homologous to the glycolytic enzyme gamma enolase. We found that autoantibodies to this protein are present predominantly in patients with NPSLE, but are rare in patients with SLE without neuropsychiatric disease and in patients with neuropsychiatric disease without underlying autoimmune disorder.

Experimental Results:

(1) 50 KDa Protein

Our initial study on neuronal targets of autoantibodies in SLE described the partial purification of a 50 kD protein associated with synaptic terminals in bovine brain. To determine the identity of the protein, further purification was accomplished. Solubilized synaptic plasma membranes were fractionated by FPLC initially by chromatography on Biogel preparative ion exchange column, followed by mono Q ion exchange separation and a final separation by phenyl-sepharose hydrophobic interaction chromatography. Fractions from the last separation were concentrated and analyzed by SDS PAGE. Immunoreactivity of the isolated protein was confirmed by Western blot analysis using serum from a patient with a defined NPSLE. The reactive band was excised from the gel matrix and digested with trypsin. The resulting peptides were separated by microcapillary reverse phase HPLC and analyzed by nano-electrospray tandem mass spectrometry. Sequenced peptides from 30 fragments showed uniquely the sequence of gamma enolase from human and other species. Two of the spectra matched alpha enolase (Table 1).

TABLE 1

Sequences of the 50 kD protein peptide
fragments matching human gamma enolase
(SEQ ID NO.s:25-41 from top to bottom)

FTANVGIQIVGDDLTVTNPK
HIAQLAGNSDLILPVPAFNVINGGSHAGNK
FTANVGIQIVGDDLTVTNPK
AAVPSGASTGIYEALELR
IVIGMDVAASEFYR
YGKDATNVGDEGGFAPNILENSEALELVK
YITGDQLGALYQDFVR
IEEELGDEAR
DYPVVSIEDPFDQDDWAAWSK
LAQENGWGVMVSHR
LGAEVYHTLK
LAQENGWGVMVSHR
DGKYDLDFK
LAMQEFMILPVGAESFR
HIAQLAGNSDLILPVPAFNVINGGSHAGNK
DGKDLDFK
AAVPSGASTGIYEALELRDGDK

(2) Immune Activity

Purified 50 kD protein was used to evaluate the reactivity of samples from 25 NPSLE patients in Western blot analysis. We found that 23 patients of this group had moderate to high reactivity with the protein. Further, 22 patients with SLE and without known neuropsychiatric disorders were tested by the same method. Only one of these patients showed low levels of reactivity with the 50 kD protein. Of the 44 patients with neuropsychiatric disease, but without SLE, only 3 patients were positive and all three patients had schizophrenia.

(3) Autoantibodies

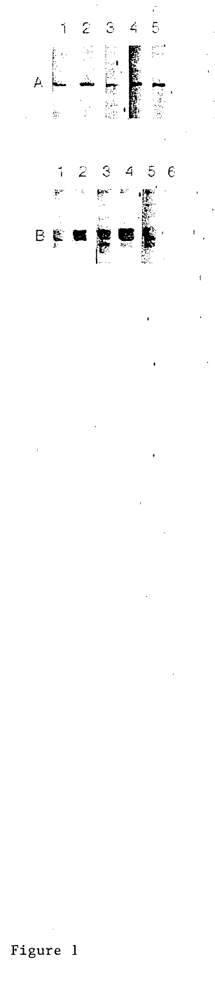

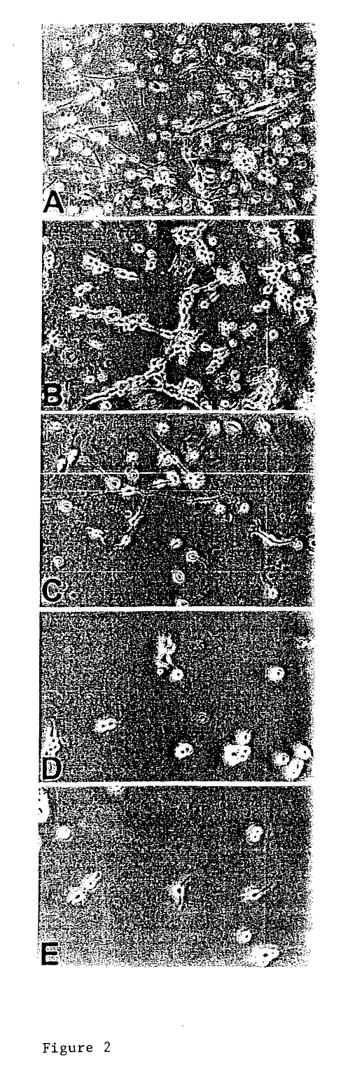

To determine whether gamma enolase indeed is the target of autoantibodies in our patients with NPSLE we tested five patients with NPSLE in Western blot analysis using commercially available preparation of gamma enolase. The reactivity of these sera was also tested with 50 kD purified protein for comparison. The data (FIG. 1) show that all five patients reacted with the commercial preparation of gamma enolase as well as the 50 kD protein.

(4) Effect on Neuronal Cells

Our original observations localized the 50 kD antigen on the surface of B104 neuroblastoma cells by immunofluorescence (6). We hypothesized that the presence of this antigen on the cell surface suggests that reactivity of autoantibodies may affect neuronal cell function and contribute to the pathogenic mechanisms in NPSLE. To evaluate the effect of autoantibodies directed to this antigen we cocultured B104 neuroblastoma cells with affinity purified autoantibodies to the 50 kD antigen. Culture of B104 neuroblastoma cells was carried out in serum free medium with an addition of affinity purified autoantibodies at 0.1 mg/ml to experimental cultures and normal human serum to control cultures. We observed that in the presence of autoantibodies, cells gradually detached from the bottom of the flask, formed free floating clumps of cells that appeared dead on day six, leaving fewer viable cells adherent to the bottom of the flask (FIG. 2). These results are consistent with an autoimmune mechanism underlying certain neuropsychiatric disorders.

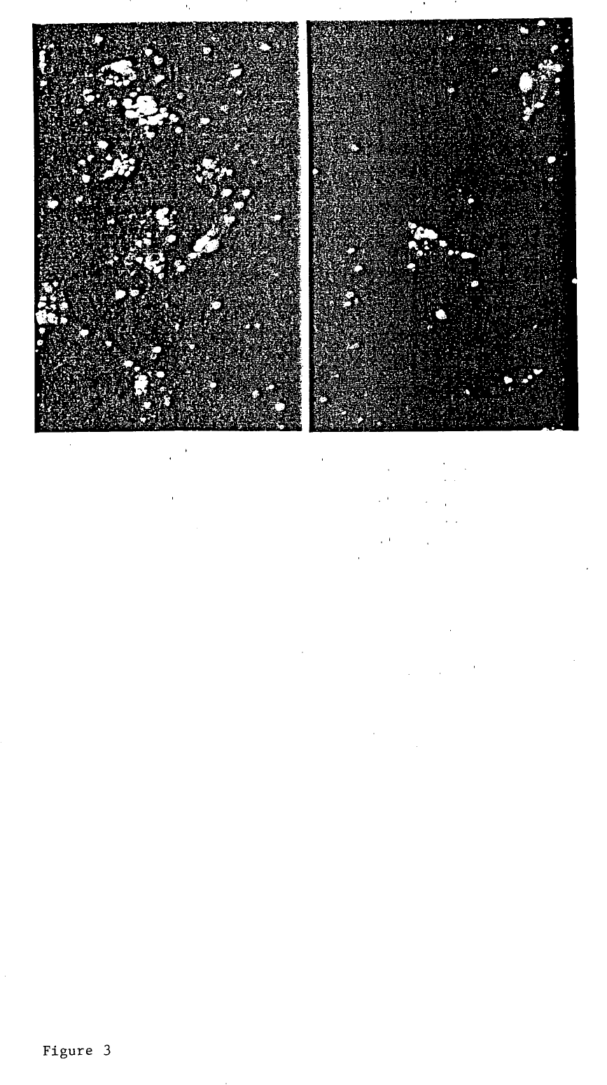

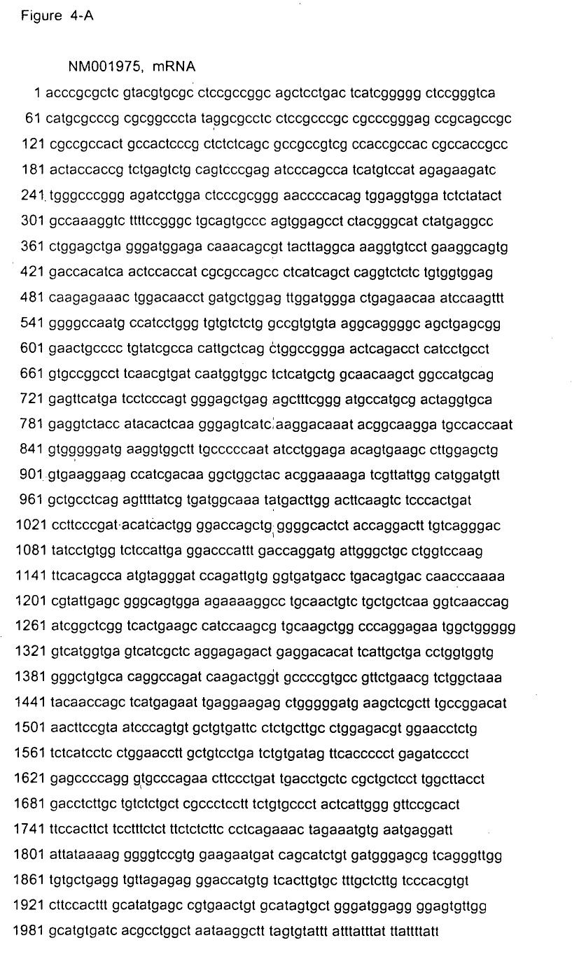

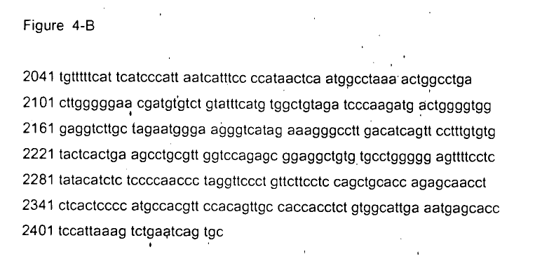

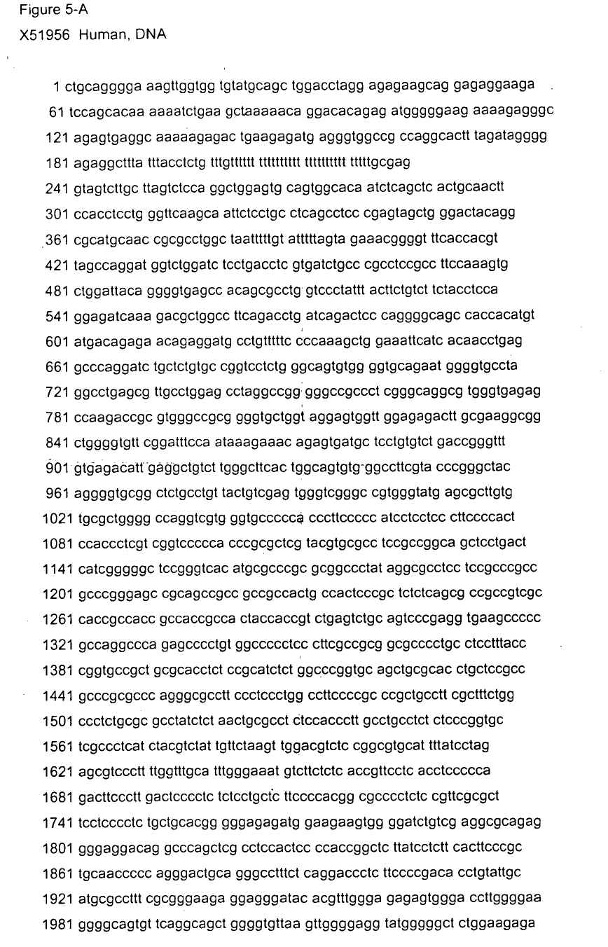

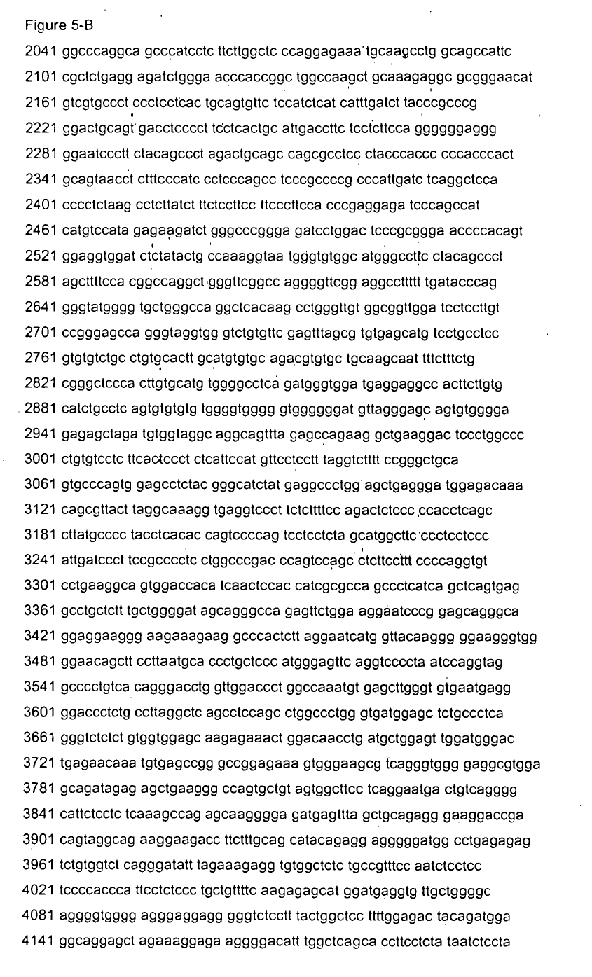

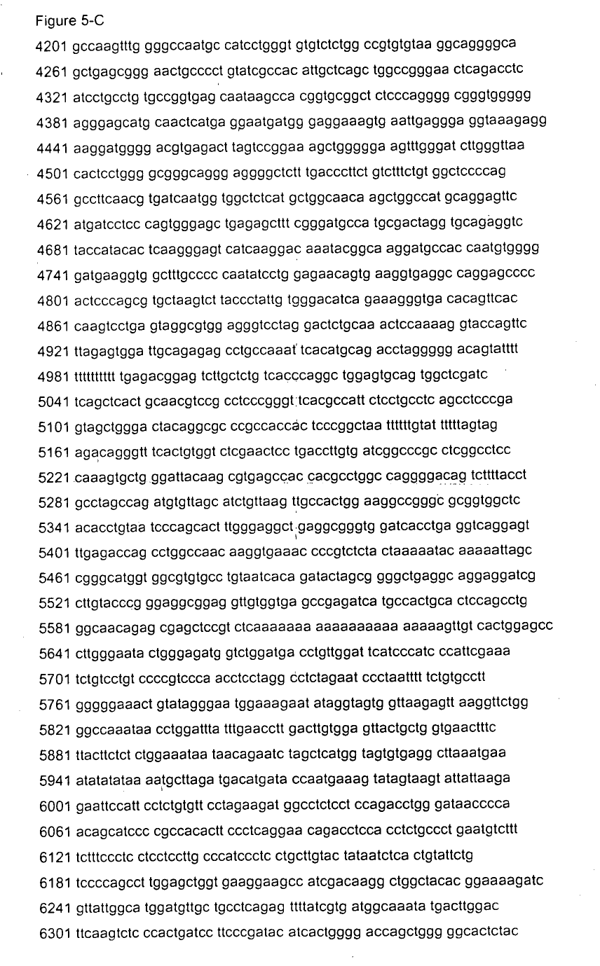

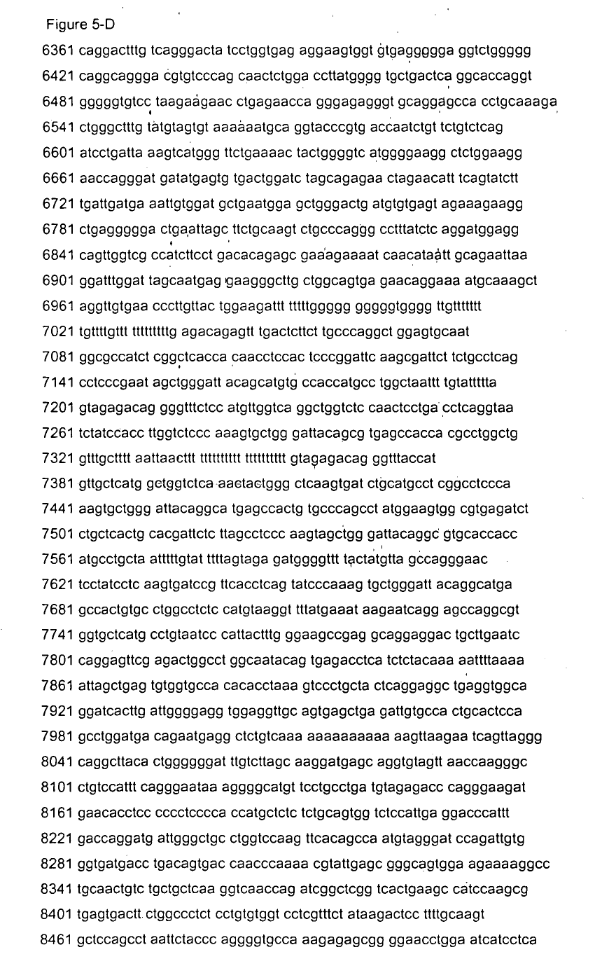

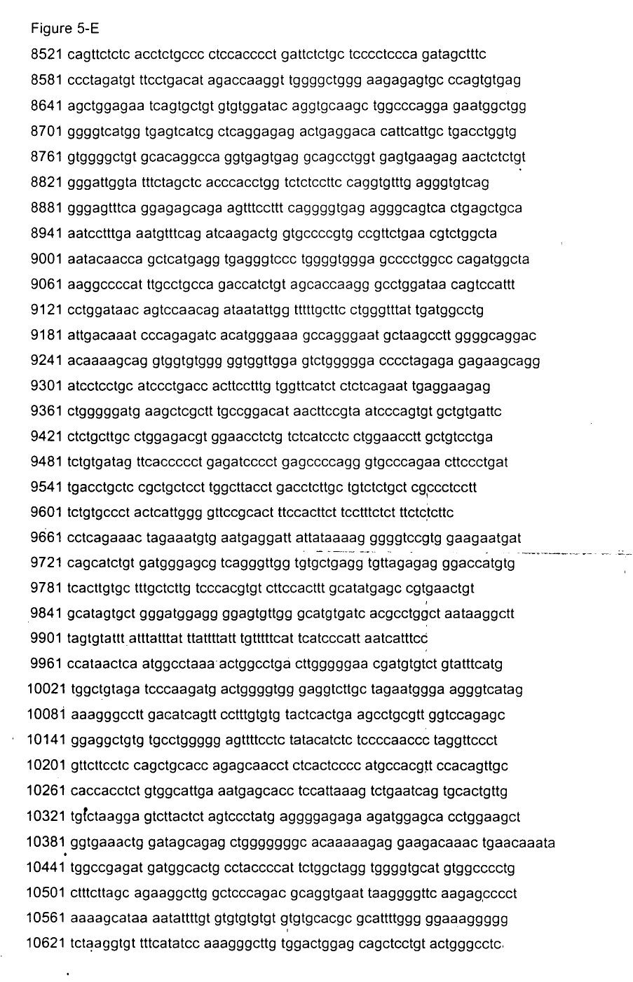

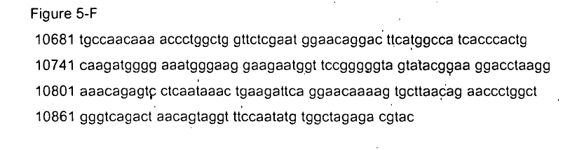

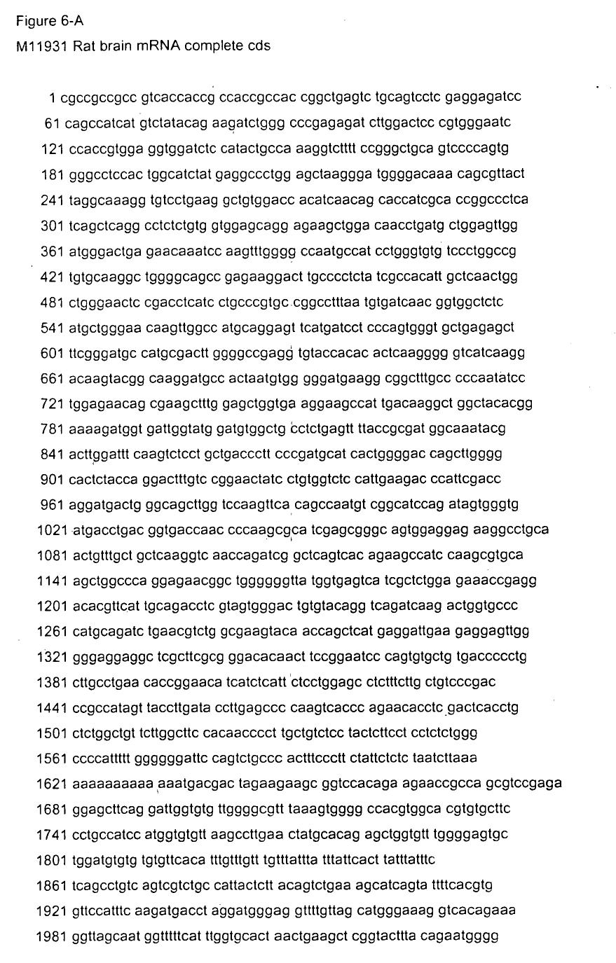

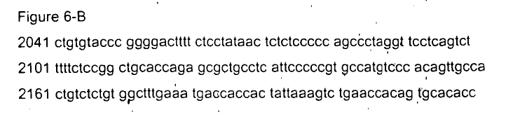

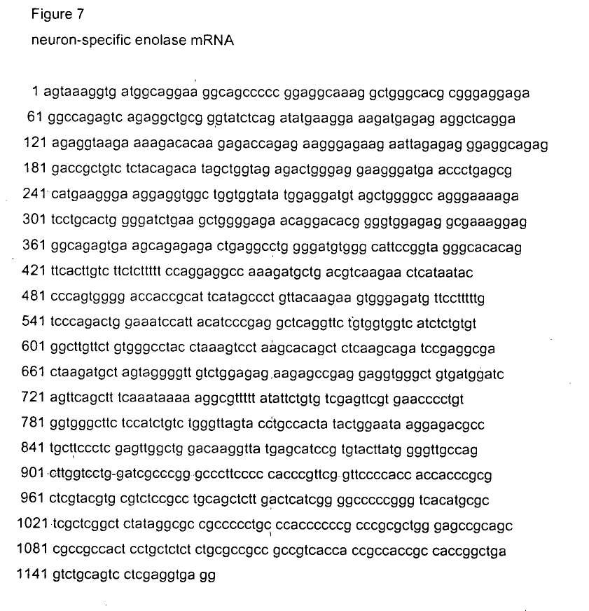

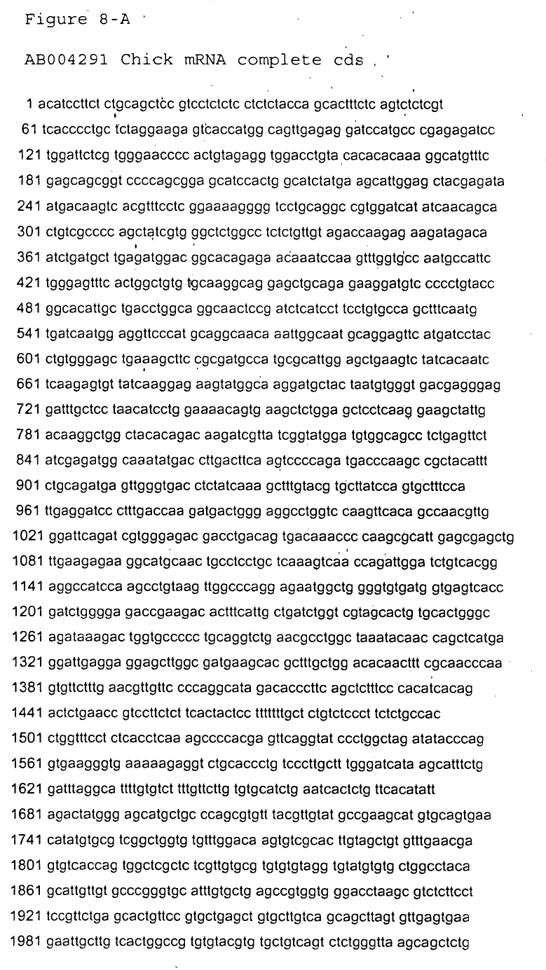

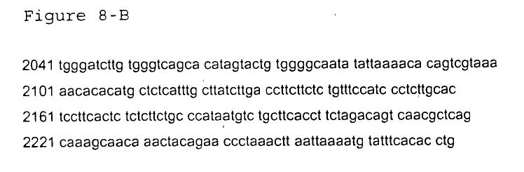

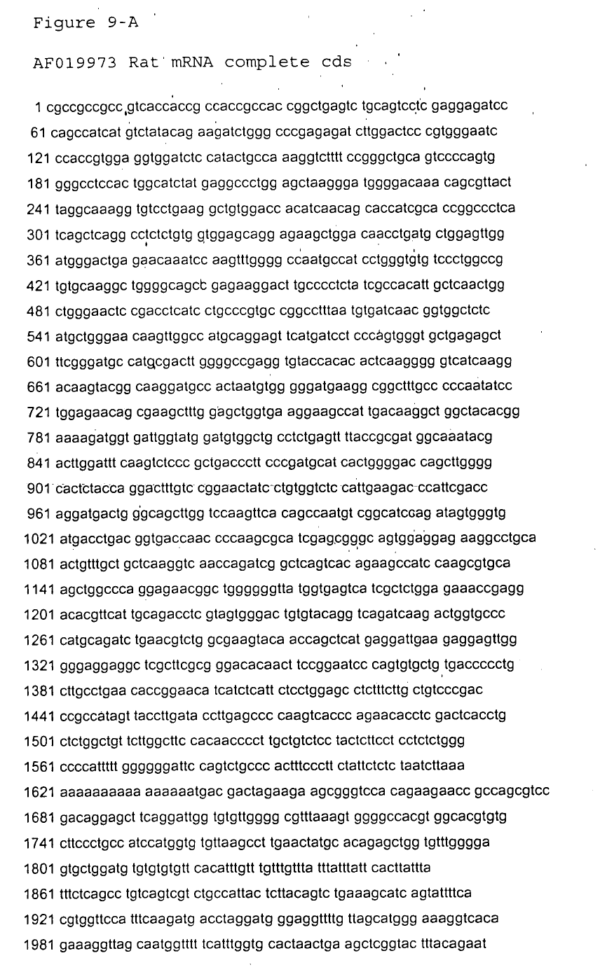

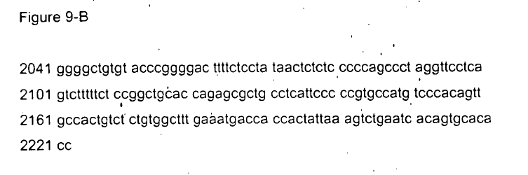

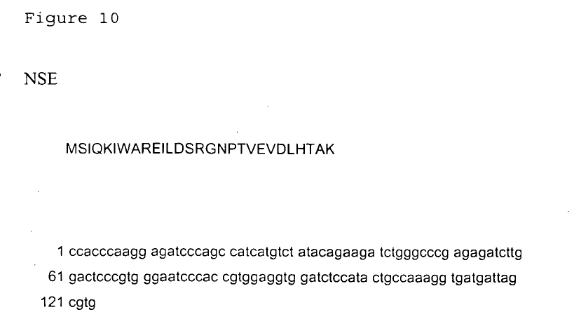

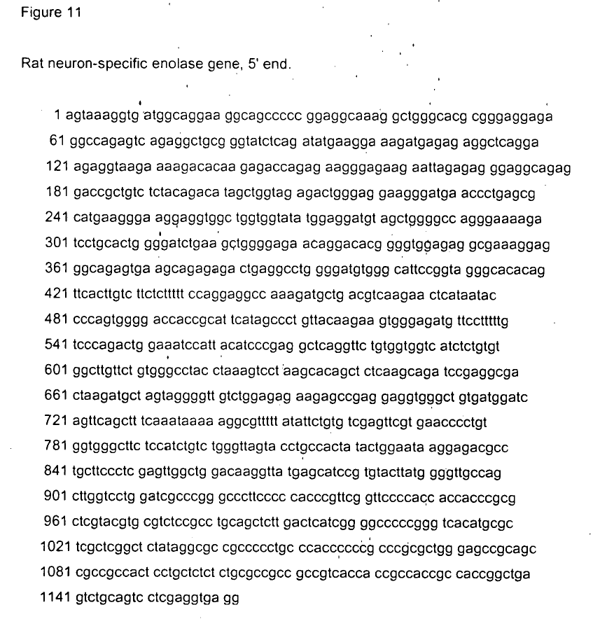

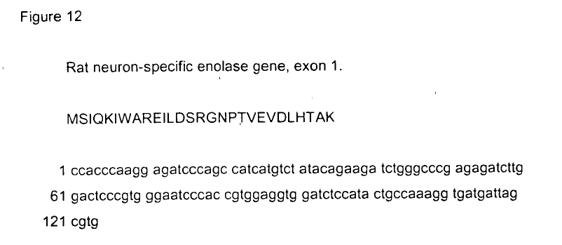

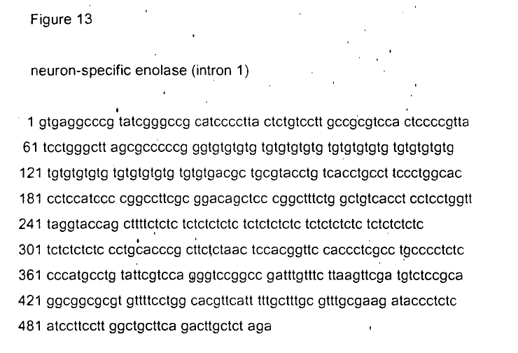

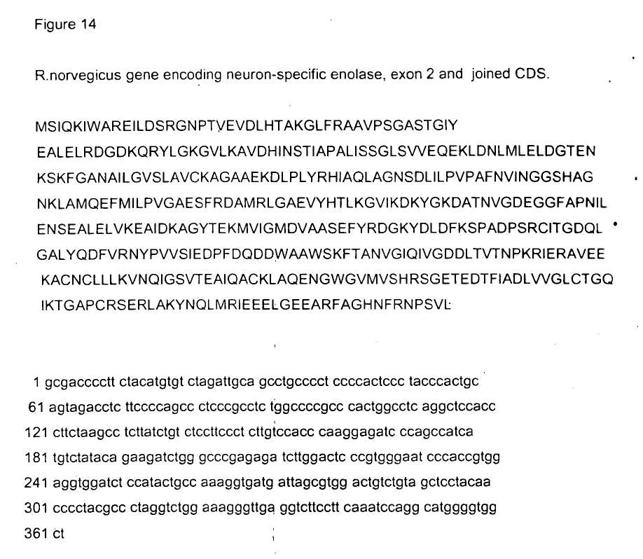

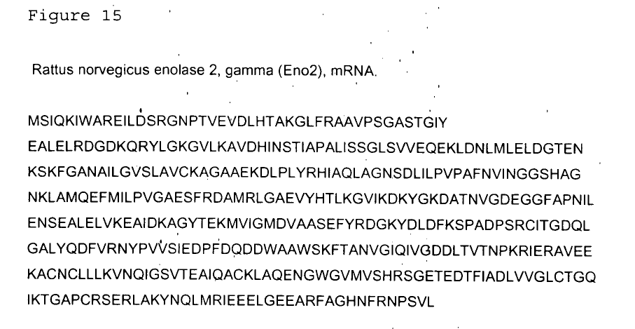

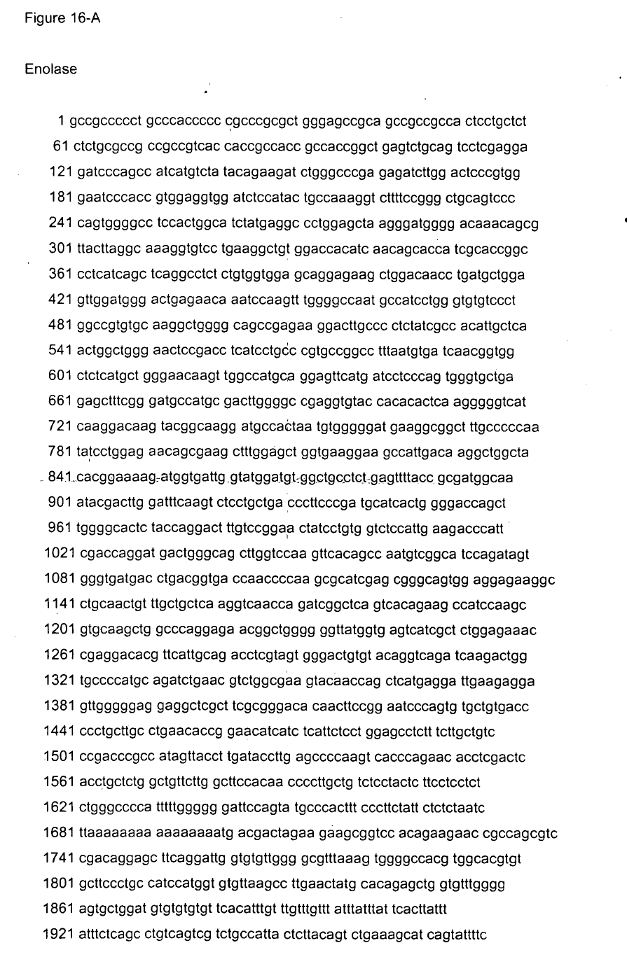

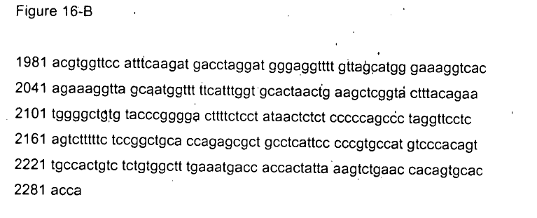

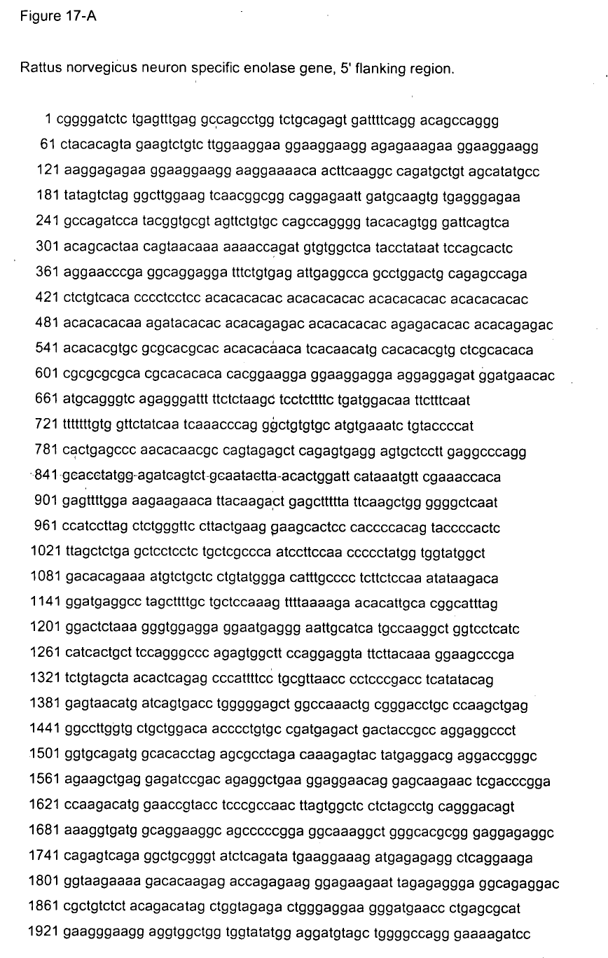

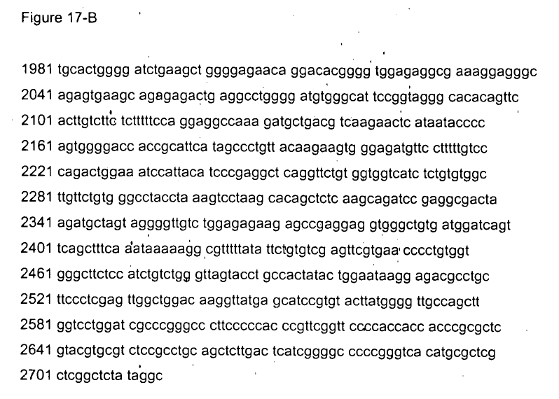

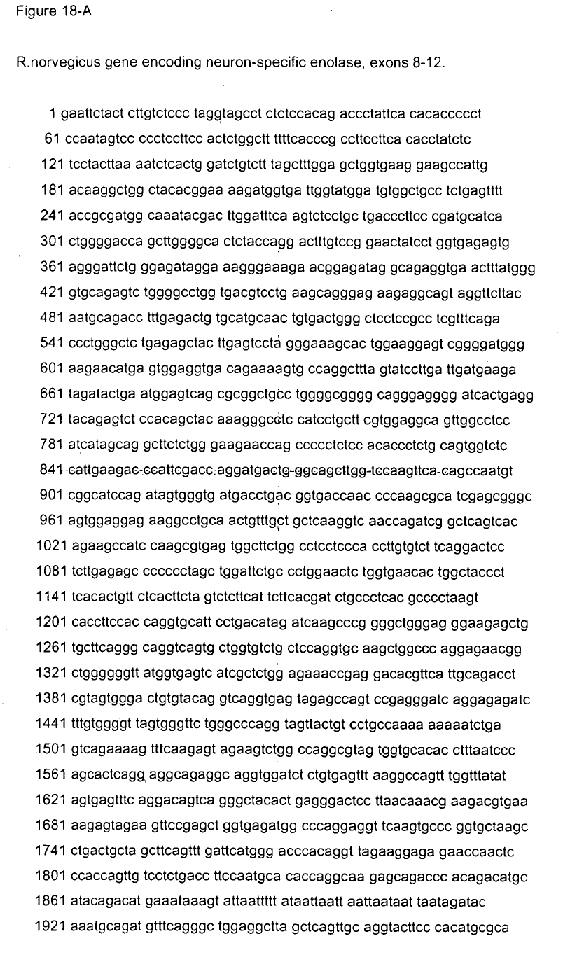

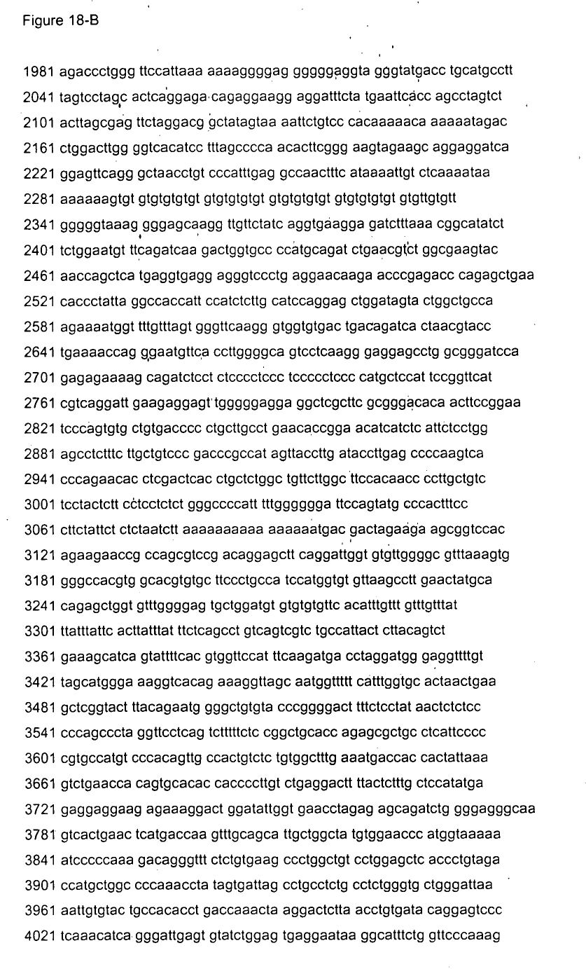

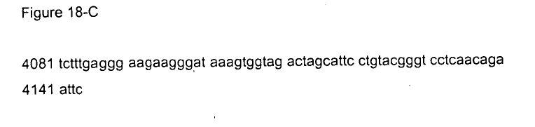

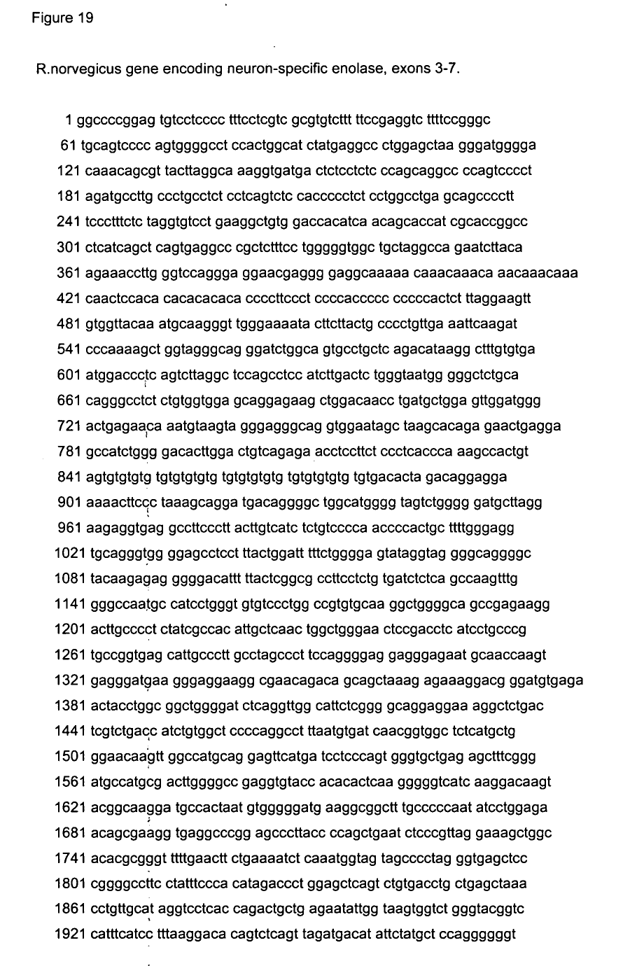

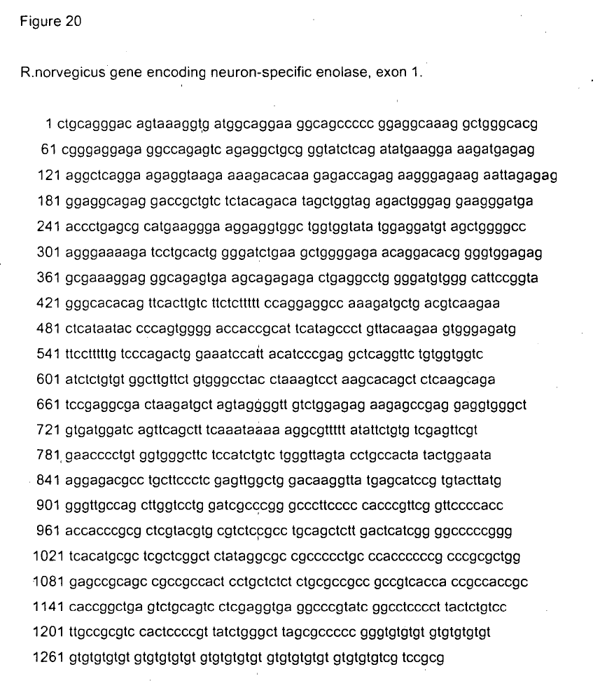

Immunofluorescent localization experiments with affinity purified autoantibodies on cells remaining attached after day six, show a different antigen localization pattern than previously described. Immunofluorescence was observed in vesicular aggregates in the perinuclear region and in the extracellular environment (FIG. 3).

Discussion

Multiple autoantibodies to membrane associated and cytoplasmic antigens other than those associated with the nucleosome have been reported in SLE. Most evident non-nucleosomal autoantibodies are those to neuronal membranes, phospholipid and the ribosomal P protein. Although these autoantibodies have been associated with NPSLE, their disease specificity remains controversial and their involvement in the pathogenic process even more so. Limited in vivo experiments indicate autoantibodies may induce neurological disease in murine animals: intraventricular injection of anti-ribosomal P protein cause behavioral changes in rats and immunization of MRL/++ mice with neuronal surface antigens from a murine neuroblastoma cell line elicits neurological disorders after 24 weeks (5). The unremarkable pathology of the CNS in NPSLE, with absence of apparent neuronal cell and tissue damage prompted the search for pathogenic mechanisms in cytokine levels in the cerebrospinal fluid (CSF), concentration levels of excitatory amino acids, changes in intracranial regional blood flow, and autoantibodies (8,9,10,11).

Autoantibodies to neuronal surface antigens have been associated with NPSLE (12). We characterized the association of autoantibodies to an unidentified synaptic membrane protein with neuropsychiatric presentations in SLE (6). The described biochemical characterization of this antigen identifies extensive homology to the glycolytic enzyme gamma-enolase. Although two spectra uniquely matched the alpha form of the enzyme, given that the gamma form is not known in bovine and the high degree of homology between the gamma and alpha forms of the isoenzyme, it is most likely that the purified 50 kD synaptic membrane protein is homologous to gamma enolase.

Enolase is a metalloenzyme of the classical glycolytic pathway that catalyses the conversion of 2-phosphoglycerate to phosphoenolpyruvate. There are three dimeric isoforms of the enzyme each encoded by one distinct gene (13,14,15). The alpha-alpha homodimer is ubiquitously distributed; it is the embryonic isoform present in most adult cells. The beta-beta isoform is specifically expressed in the differentiated striated muscle (16). Although the gamma-gamma isoform is shown to be a neuron specific enolase (NSE), present only in differentiated neuronal cells, and the alpha-alpha isoform is found to be restricted to glial cells, transcription of both alpha and gamma genes can be detected in many neurons (17). The neuron specific expression of the gamma enolase enzyme may impart organ specific rather than systemic self-reactivity, which maybe relevant to NPSLE. In mammals the tissue specific isoforms are structurally highly conserved in terms of primary and tertiary structure, showing 82% amino acid sequence identity between the three isoforms and are believed to be functionally very similar (18). The short variable regions in each subunit are localized on the surface of the molecule and are postulated to impart specific subcellular macromolecular targeting modulated by posttranslational modifications. These protein regions may confer differential immunogenicity of the subunits.

It has been shown that cell specific isoforms differ in their properties. NSE is the most acidic isoform with higher tolerance to chloride ions as well as higher thermal stability. These properties are believed to be molecular adaptation to hypoxic, inflammatory or oxidative stress events (19). It is present in purified synaptic plasma membranes and synaptic vesicles from bovine and rat brains, and in neuroblastoma cells as part of a tightly bound complex of dichlorophenol-indophenol oxidoreductase, glyceraldehyde-3-phosphate dehydrogenase isoform, TOAD64, aldolase and the heat shock70 protein (20,21,22). It is postulated that this protein complex may have a function in membrane fusion and cellular responses to oxidative stress and it has been shown by the same investigators that antibodies to the various components of the protein complex can inhibit their enzymatic activity.

NSE is present normally at low levels in the cerebrospinal fluid (23). These low concentrations may contribute to a continuous exposure of T and B cells to the enzyme, assuring immunological tolerance. A significant increase over normal levels of NSE in the cerebrospinal fluid is found in various pathological conditions such as cerebral trauma (24), brain tumors (25), acute ischemic stroke (26), Creutzfeld-Jacob disease (27), Lyme neuroborreliosis (28), bacterial meningitis (29), dementia (30), Alzheimer's disease (31), and after cardiac arrest (32). In fact, increased levels of NSE in the cerebrospinal fluid is considered reliable and specific marker of neuronal damage (33,34). Sustained elevation of the concentration of NSE in the cerebrospinal fluid may contribute to loss of tolerance to this enzyme. This may be of particular significance for patients with an autoimmune disease, where there is an intrinsic problem with self-recognition. Therefore, in patients with autoimmune disease sustained increased levels of cerebrospinal concentrations of NSE may trigger T and B cell stimulation and production of autoantibodies. In support to this hypothesis is the recent observation that lupus prone NZB mice are characterized by overexpression of mRNA for the alpha form of enolase specifically in B cells, and that soluble alpha enolase has B cell stimulatory role (35).

Autoantibodies to members of the enolase family are present in various autoimmune and inflammatory diseases: The alpha form of enolase is a target of autoantibodies in 40% of clinically active vasculitis patients with antineutrophil cytoplasmic antibodies (ANCA) (36,37). ANCA reactive antibodies from patients with primary sclerosing cholangitis and Crohn's disease bind alpha-enolase, but these reactivities have been found in less than 20% of this patient group (38). Autoantibodies to alpha enolase have also been reported in 29% of patients with primary billiary sclerosis, in 32% of patients with autoimmune hepatitis (39), in 50% of patients with endometriosis (40), in 30% of patients with systemic sclerosis (41) and in 30% of patients with mixed cryoglobulinemia and nephritis (42). Patients with renal involvement and autoimmune disease show higher incidence of autoantibodies to alpha enolase. 69% of patients with primary membranous nephropathy and 58% of patients with membranous nephropathy secondary to rheumatoid arthritis or SLE show serum autoantibodies to alpha-enolase. Pratesi et al. have reported anti alpha-enolase antibodies in the serum of 27% of SLE patients with active nephritis (43).

Autoantibodies to enolases may impair cellular function via multiple pathways. There is increasing evidence that the enolase isoenzymes play a functional role separate from glycolysis in the cell and that specific cellular compartmentalization may impart functional specialization of the isoenzymes. In the case of alpha enolase the evidence for multifunctional activity is well documented. Alpha enolase is a potential tumor suppressor (44), a possible plasminogen receptor (45) with surface expression on important inflammatory cells such as neutrophils, T cells, B cells and monocytes, as well as endothelial cells (46). The enzyme is also shown to function as a heat shock protein (47) and a transcription factor for paramyxovirus at a replication site in the cell (48).

There is increasing evidence that NSE has neurotrophic and survival factor properties for embryonic rat neocortical, mesencephalic, and spinal cord neurons (49), as well as for photoreceptor neurons (50,51). Circulating autoantibodies may interfere with the survival factor properties of NSE. Our coculture experiments show that autoantibodies to NSE affect the survival of B104 neuroblastoma cells and this effect is associated with antigen redistribution. Supporting evidence for this premise is also provided by the data of Maruyama et al. (52), who observed that intravitreal administration of purified anti-NSE antibody induces apoptotic cell death in retinal ganglion cells.

There is accumulating evidence that autoantibodies to glycolytic enzymes may be a contributing factor to the pathogenic mechanisms of autoimmune diseases. Recently, Schaller et al. (53) described T and B cell stimulation by the glycolytic enzyme glucose-6-phosphate isomerase (GPI), a multifunctional enzyme with cytokine activity, neuronal survival factor activity, autocrine motility factor activity and maturation factor activity. The authors linked serum and synovial fluid autoantibodies against GPI to maintaining RA in humans. In the K/BxN TCR—transgenic mouse, which is a model for RA, the disease is initiated by T cell recognition of GPI and pathology of the disease is attributed to the autoantibodies to GPI (54). As stated above Babu et al (35) established that enolase has a B cell stimulatory effect. Further studies on the association of NPSLE with autoantibodies to NSE may provide similar evidence.

Materials and Methods

Patient Population:

The patient population consisted of 25 patients with SLE without neuropsychiatric manifestations, 20 patients with NPSLE, 44 patients with non-SLE neuropsychiatric disease, including 6 patients with amyotrophic lateral sclerosis, 15 patients with Alzheimer's disease, 13 patients with bipolar disorder and 10 patients with schizophrenia.

Purification of the 50 kD Antigen:

Synaptic plasma membranes were obtained from two bovine brains using the method of Jones and Matues (7). Synaptosomal proteins solubilized with TRITON X-100 were applied on a Pharmacia biogel preparative column, and fraction eluted with 10-500 mM phosphate buffer gradient at pH 6.8 containing 0.1% octylglucoside. Fractions were screened by Western blot and for the presence of the 50 kD protein using serum from a patient with NPSLE. Positive fractions were dialyzed in 10 mM Tris buffer, pH 8.0 containing proteolytic inhibitors. After dialysis these fractions were loaded on a Pharmacia Mono Q column equilibrated with the same buffer. Elution was performed with a 10-500 mM KCl gradient. Eluted fractions were screened by Western blot and positive fractions were pooled and loaded on a Pharmacia phenyl sepharose column. Ammonium sulfate gradient was used for elution. Proteins eluted from the column were separated by SDS PAGE. The immunopositive band was excised. The protein was proteolytically digested and peptide fragments were separated by microcapillary reverse-phase HPLC. Fractions were analyzed by nanospray tandem mass spectrometry.

Affinity Purification of Human Sera:

Purified 50 kD protein was immunoblotted on nitrocellulose membranes and the 50 kD area was cut into strips. Serum from a patient with well characterized NPSLE was incubated with the membranes overnight at 4° C. Membranes were washed with Tris buffered saline, containing 0.1% TWEEN and eluted with 0.1M glycine buffer, pH2.3. After neutralization in 0.5M Tris buffer pH 8.8 the antibodies were concentrated and dialyzed in phosphate buffered saline.

### REFERENCES

1. Hochberg M C. Updating the American College of Rheumatology revised criteria for classification of systemic lupus erythematosus. Arthritis Rheum. 1997; 40: 1725.
2. ACR ad hock committee on neuropsychiatric lupus nomenclature. The American College of Rheumatology nomenclature and case definitions for neuropsychiatric lupus syndromes. Arthritis Rheum. 1999; 42: 599-608.
3. Tzioufas A G, Tzortzakis N G, Panou-Pomonis E, Boki K A, Sakarellos-Daitsiotis M, Sakarellos C, Moutsopoulos H M. The clinical relevance of antibodies to ribosomal-P common epitope in two targeted systemic lupus erythematosus populations: a large cohort of consecutive patients and patients with active central nervous system disease. Ann. Rheum. Dis. 2000; 59: 99-104.
4. Isshi K, Hirohata S. Differential roles of the anti-ribosomal P antibody and antineuronal antibody in the pathogenesis of central nervous system involvement in systemic lupus erythematosus. Arthritis Rheum. 1998; 41: 1819-27.
5. Brey R L, Sakic B, Szechtman H, Denburg J. Animal models for nervous system disease in systemic lupus erythematosus. Ann. NY Acad. Sci. 1997; 823: 97-109.
6. Hanson V G, Horowitz M, Rosenblum D, Spiera H, Puszkin S. Systemic lupus erythematosus patients with central nervous system involvement show autoantibodies to a 50 kD neuronal antigen. J. Exp. Med. 1992; 176: 565-573.
7. Jones D H, Matus A I. Isolation of synaptic plasma membrane from brain by combined flotation-sedimentation density gradient centrifugation. Biochim Biophys Acta 1974; 356: 276-287.
8. Hanly J G, Welsh N M, Sangalang V. Brain pathology in systemic lupus erythematosus. J. Rheumatol. 1992; 19: 732-741.
9. Tomita M, Holman B J, Williams L S, Pang K C, Santoro T J. Cerebellar dysfunction is associated with overexpression of proinflammatory cytokine genes in lupus. J. Neurosci. Res. 2001; 64: 26-33.
10. Dean G S, Tyrell-Price J, Crawley E, Isenberg D A. Cytokines in systemic lupus erythematosus. Ann. Rheum. Dis. 2000; 59: 243-251.
11. Alosachie I J, Terryberry J W, Mevorach D, Chapman Y, Lorber M, Torre D, Youinou P, Peter J B, Shoenfeld Y. Central nervous system (CNS) involvement in SLE. The diagnostic role of antibodies to neuronal antigens. Clin. Rev. Allergy Immunol. 1998; 16: 275-84.
12. DeGiorgio L A, Konstantinov K N, Lee S C, Hardin J A, Volpe B T, Diamond B. A subset of lupus anti-DNA antibodies cross-reacts with the NR2 glutamate receptor in systemic lupus erythematosus. Nat. Med. 2001; 7: 1189-93.
13. Cook P J L, Hamerton J L. Report on the committee on the genetic constitution of chromosome 1. Fifth International Workshop on Human Gene Mapping. Cytogenet. Cell Genet. 1979; 25: 9-20.
14. Feo S, Oliva D, Barbiery G, Xu W, Fried M, Galliango A. The gene for the muscle specific enolase is on the short arm of chromosome 16. Genomics 1990; 6: 192-194.
15. Craig S P, Day I N M, Thompson R J, Craig I W. Localization of neuron-specific enolase (ENO2) to 12p13. Cytogenet. Cell. Genet. 1990; 54: 71-73.
16. Merculova T, Lucas M, Jabet C, Lamande N, Rouzeau J D, Gros F, Lazar M, Keller A. Biochemical characterization of the mouse muscle-specific enolase: developmental changes in electrophoretic variants and selective binding to other proteins. Biochem. J. 1997; 323: 791-80.
17. Vinores S A, Herman M M, Rubinstein L J, Marangos P J. Electron microscopic localization of neuron-specific enolase in rat and mouse brain. J. Histochem. Cytochem. 1984; 32: 1295-302.
18. McAleese S M, Dunbar B, Fothergill J E, Hinks L J, Day J N, Complete amino acid sequence of the neuron specific gamma isozyme of enolase from human brain and comparison with the non-neuronal alpha form. Eur. J. Biochem. 1988; 178: 413-417.
19. Marangos P J, Schmechel D E. Neuron specific enolase, a clinically useful marker for neurons and neuroendocrine cells. Ann. Rev. Neurosci. 1987; 10: 269-295.
20. Leung T K, Hall C, Monfries C, Lim L. Trifluoperazine activates and releases latent ATP-generating enzymes associated with the synaptic plasma membrane. J. Neurochem. 1987; 49: 232-8.
21. Tamarit J, Cabiscol E, Ros J. Identification of the major oxidatively damaged proteins in Escherichia coli cells exposed to oxidative stress. J. Biol. Chem. 1998; 273: 3027-3032.
22. Bulliard C, Zurbriggen R, Tornare J, Faty M, Dastoor Z, Dreyer J L. Purification of a dichlorophenol-indophenol oxidoreductase from rat and bovine synaptic membranes: tight complex association of a glyceraldehydes-3-phosphate dehydrogenase isoform, TOAD64, gamma enolase and aldolase C. Biochem. J. 1997; 324: 555-563.
23. Reiber H. Dynamics of brain-derived proteins in cerebrospinal fluid. Clin. Chim. Acta 2001; 310: 173-86.
24. Dauberschmidt R, Marangos P J, Zinsmeyer J, Bender V, Klages G, Gross J. Severe head trauma and the changes of concentration of neuron-specific enolase in plasma and in cerebrospinal fluid. Clin. Chim. Acta 1983 15(131): 165-70.
25. Boiardi A, Munari L, Silvani A, Solero C L, Bombardieri E. Neuron specific enolase (NSE) and thymidine kinase (TK) as markers in biological fluids of brain tumor patients. Ital. J. Neurol. Sci. 1990; 11: 359-66.
26. Missler U, Wiesmann M, Friedrich C, Kaps M. S-100 protein and neuron-specific enolase concentrations in blood as indicators of infarction volume and prognosis in acute ischemic stroke. Stroke. 1997; 28: 1956-60.
27. Kohira I, Tsuji T, Ishizu H, Takao Y, Wake A, Abe K, Kuroda S. Elevation of neuron-specific enolase in serum and cerebrospinal fluid of early stage Creutzfeldt-Jakob disease. Acta Neurol. Scand. 2000; 102: 385-7.
28. Dotevall L, Hagberg L, Karlsson J E, Rosengren L E. Astroglial and neuronal proteins in cerebrospinal fluid as markers of CNS involvement in Lyme neuroborreliosis. Eur. J. Neurol. 1999; 6: 169-78.
29. Inoue S, Takahashi H, Kaneko K. The fluctuations of neuron-specific enolase (NSE) levels of cerebrospinal fluid during bacterial meningitis: the relationship between the fluctuations of NSE levels and neurological complications or outcome. Acta. Paediatr. Jpn. 1994; 36: 485-8.
30. Nooijen P T, Schoonderwaldt H C, Wevers R A, Hommes O R, Lamers K J. Neuron-specific enolase, S-100 protein, myelin basic protein and lactate in CSF in dementia. Dement. Geriatr. Cogn. Disord. 1997; 8: 169-73.
31. Parnetti L, Palumbo B, Cardinali L, Loreti F, Chionne F, Cecchetti R, Senin U. Cerebrospinal fluid neuron-specific enolase in Alzheimer's disease and vascular dementia. Neurosci. Lett. 1995; 183: 43-5.
32. Kaukinen L, Porkkala H, Kaukinen S, Pehkonen E, Karkela J, Aaran R K, Tarkka M. Release of brain-specific creatine kinase and neuron-specific enolase into cerebrospinal fluid after hypothermic and normothermic cardiopulmonary bypass in coronary artery surgery. Acta Anaesthesiol. Scand. 2000; 44: 361-8.
33. Hans P, Bonhomme V, Collette J, Moonen G. Neuron-specific enolase as a marker of in vitro neuronal damage. Part I: Assessment of neuron-specific enolase as a quantitative and specific marker of neuronal damage. J. Neurosurg. Anesthesiol. 1993; 5: 111-6.
34. Schmitt B, Bauersfeld U, Schmid E R, Tuchschmid P, Molinari L, Fanconi S, Bandtlow C. Serum and CSF levels of neuron-specific enolase (NSE) in cardiac surgery with cardiopulmonary bypass: a marker of brain injury? Brain Dev. 1998; 20: 536-9.
35. Babu J S, Sun T, Xu L, Data S K. B cell stimulatory effects of alpha enolase that is differentially expressed in NZB mouse B cells. Clin. Immunol. 2002; 104: 293-304.
36. Moodie F D, Leaker B, Cambridge G, Totty N F, Segal A W. Alpha enolase: A novel cytosolic autoantigen in ANCA positive vasculitis. Kidney Int. 1993; 43: 675-681.
37. Orth T, Kellner R, Diekmann O, Faust J, Meyer zum Bueschenfelde K H, Mayet W J. Identification and characterization of autoantibodies against catalase and alpha enolase in patients with primary sclerosing cholangitis. Clin. Exp. Immunol. 1998; 112: 507-515.
38. Roozendaal C, Zhao M H, Horst G, Lockwood C M, Kleibbeuker J H, Limburg P C, Nelis G I. Catalase and alpha enolase: Two novel granulocyte autoantigens in inlamatory bowel disease. Clin. Exp. Immunol. 1998; 112: 10-16.
39. Akisawa N, Maeda T, Iwasaki S, Onishi S. Identification of a autoantibody against alpha enolase in primary billiary cerrhosis. J. Hepathol. 1997; 26: 845-851.
40. Walter M, Burg H, Leidenberger F A, Schweppe K W, Northermann W. Autoreactive epitopes within human alpha enolase and their recognition by sera from patients with endometriosis. J. Autoimmun. 1995; 8: 931-945.
41. Wakui H, Imai H, Komatzuda A, Miura A B. Circulating antibodies against alpha enolase in patients with primary membranious nephropathy. Clin. Exp. Immunol. 1999; 118:445-450.
42. Sabbatini A, Dolher M P, Marchini B, Chimenti D, Moscato S, Pratesi F, Bombardieri S, Migliorini P. Alpha enolase is a renal specific antigen associated with kidney involvement in mixed cryoglobulemia. Clin. Exp. Rheumatol. 1997; 15: 655-658.
43. Pratezi F, Moscato S, Sabbatini A, Chimenti D, Bombardieri S, Migliorini P. Auoantibodies specific for alpha-enolase in systemic autoimmune disorders. J. Rheumatol. 2000; 27: 109-115.
44. Feo S, Arcuri D, Piddini E, Passantino R, Giallongo A. ENO1 gene product binds to the c-myc promoter and acts as a transcriptional repressor: ralationship with Myc promoter-binding protein 1 (MBP-1). FEBS Letters 2000; 473: 47-52.
45. Arza B, Felez J, Lopez-Alemany R, Miles L. Identification of an epitope of alpha-enolase (a candidate plasminogen receptor) by phage display. Thromb Haemost 1997; 78: 1097-1103.
46. Yosefi S, Cooper P R, Mueck B, Potter S L, Jarai G. cDNA representational differences analysis of humam neutrophills stimulated by GM-CSF. Biochem. Biophys. Res. Com. 2000; 277: 401-409.
47. Iida H, Yahara I. Yeast heat-shock protein is an isoprotein of enolase. Nature 1985; 315: 688-689.
48. Ogino T, Yamadera T, Takashi N, Imajoh S, Mizumoto K. Enolase, a cellular glycolytic enzyme, is required for efficient transcription of sendai virus genome. Biophys. Biochem. Res. Comm. 2001; 285: 447-455
49. Takei T, Kondo J, Nagaike K, Ohsawa, Kato K, Kohsaka S. Neuronal survival factor from bovine brain is identical to neuron-specific enolase. J. Neurochem. 1991; 57: 1178-1184.
50. Li A., Lane W S, Johnson L V, Chader G J, Tombran-Tink J. Neuron-specific enolase: a neuronal survival factor in the retinal extracellular matrix. J. Neurosci. 1995; 15: 385-393.
51. Hattori T, Takei N, Mizuno Y, Kato K, Kohsaka S. Neurotrophic and neuroprotective effects of neuron-specific enolase on cultured neurons from embryonic rat brain. Neurosci. Res. 1995; 21: 191-198.
52. Maruyama I, Nakazawa M, Ohguro H. Autoimmune mechanisms in molecular pathology of glaucomatous optic neuropathy. Nippon Ganka Gakkai Zasshi. 2001; 105: 205-12.
53. Schaller M, Burton D R, Ditzel H J. Autoantibodies to GPI in rheumatoid arthritis: linkage between an animal model and human disease. Nat. Immunol. 2001; 2: 746-53.
54. Matsumoto I, Staub A, Benoist C, Mathis D. Arthritis provoked by linked T and B Cells recognition of a glycolytic enzyme. Science 2001; 286: 1732.

1. A method of assessing the likelihood that a patient is suffering from neuropsychiatric systemic lupus erythematosus which comprises:
a) obtaining a fluid sample from the subject;
b) contacting the sample with an agent which forms a complex with an autoantibody to a protein comprising consecutive amino acids having the sequence set forth in SEQ ID NO:21 or 23, under conditions permitting any such autoantibody present in the sample to complex with the agent; and
c) detecting the presence of any autoantibody-agent complex formed in step (b);
wherein the detection of autoantibody-agent complex in step (c) indicates that the patient is likely suffering from neuropsychiatric systemic lupus erythematosus.

2. The method of claim 1, wherein the fluid sample comprises sera, plasma, urine, saliva, synovial fluid, cerebro-spinal fluid, or lymph.

3. The method of claim 1, wherein the agent is an antibody or fragment thereof which binds to the autoantibody and is labeled with a detectable marker.

4. The method of claim 1, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

5. The method of claim 4, wherein the protein or the immunogenic fragment thereof is labeled with a detectable marker.

6. The method of claim 5, wherein the detectable marker is a radioisotope, a chromophore, a biomolecule, a fluorophore, a radiolabeled molecule, a dye, an affinity label, an antibody, biotin, streptavidin, a metabolite, a mass tag, or a dextran.

7. The method of claim 1, wherein the detecting in step (c) comprises contacting the autoantibody-agent complex with a second antibody which binds to the autoantibody-agent complex and is labeled with a detectable marker.

8. The method of claim 1, further comprising determining the amount of complex formed in step (b) and comparing such amount with a standard, wherein a greater amount of complex formed in step (b) than in the standard indicates that the subject is likely suffering from neuropsychiatric systemic lupus erythematosus.

9. The method of claim 9, wherein the standard is a fluid sample comprising sera, plasma, urine, saliva, synovial fluid, cerebro-spinal fluid, or lymph from a patient not suffering from neuropsychiatric systemic lupus erythematosus.

10. A method of assessing the likelihood that a patient is suffering from neuropsychiatric systemic lupus erythematosus which comprises:
a) providing a solid support to which an agent which forms a complex with an autoantibody to a protein comprising consecutive amino acids having the sequence set forth in SEQ ID NO:21 or 23, under conditions permitting any such autoantibody present in the sample to complex with the agent is bound;
b) contacting the solid support from (a) with a fluid sample from the subject;
c) removing any of the autoantibody which is not bound to the solid support; and
d) detecting the presence of autoantibody bound to the solid support,
wherein the detection of autoantibody bound to the solid support in step (d) indicates that the patient is likely suffering from neuropsychiatric systemic lupus erythematosus.

11. The method of claim 10 wherein the agent is a protein comprising consecutive amino acids having the sequence set forth in SEQ ID NO:21, SEQ ID NO:22, SEQ ID NO:23, SEQ ID NO:24, or an immunogenic fragment thereof and wherein the detecting of the presence of autoantibody bound to the solid support with an antibody which binds to the autoantibody is labeled with a detectable marker.

12. The method of claim 11, wherein the fluid sample comprises sera, plasma, urine, saliva, synovial fluid, cerebro-spinal fluid, or lymph.

13. The method of claim 11, wherein the detectable marker is a radioisotope, a chromophore, a biomolecule, a fluorophore, a radiolabeled molecule, a dye, an affinity label, an antibody, biotin, streptavidin, a metabolite, a mass tag, or a dextran.

14. The method of claim 11, further comprising determining the amount of complex formed in step (b) and comparing such amount with a standard, wherein a greater amount of complex formed in step (b) than in the standard indicates that the subject is likely suffering from neuropsychiatric systemic lupus erythematosus.

15. The method of claim 14, wherein the standard is a fluid sample comprising sera, plasma, urine, saliva, synovial fluid, cerebro-spinal fluid, or lymph from a patient not suffering from neuropsychiatric systemic lupus erythematosus.

16. A diagnostic kit which comprises a container comprising an agent which forms a complex with an autoantibody to a protein comprising consecutive amino acids having the sequence set forth in SEQ ID NO:21 or 23, which agent is labeled with a detectable marker.

17. The kit of claim 16, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

18. The method of claim 16, wherein the agent is an antibody or fragment thereof which binds to the autoantibody and is labeled with a detectable marker.

19. A diagnostic kit which comprises a container comprising a solid support to which an agent which forms a complex with an autoantibody to a protein comprising consecutive amino acids having the sequence set forth in SEQ ID NO:21 or 23 is bound, which agent is labeled with a detectable marker.

20. The kit of claim 19, wherein the agent comprises a protein comprising consecutive amino acids having the sequence set forth in SEQ ID NO:21, SEQ ID NO:22, SEQ ID NO:23, SEQ ID NO:24, or an immunogenic fragment thereof.

21. The method of claim 19, wherein the agent is an antibody or fragment thereof which binds to the autoantibody and is labeled with a detectable marker.
